(19) United States
(12) Patent Application Publication
(10) Pub. No.: US 2025/0034427 A1
(43) Pub. Date: Jan. 30, 2025

(54) CHEMICAL-RESISTANT PROTECTIVE FILM-FORMING COMPOSITION HAVING CATECHOL GROUP

(71) Applicant: NISSAN CHEMICAL CORPORATION, Tokyo (JP)

(72) Inventors: Shun KUBODERA, Toyama-shi (JP); Tokio NISHITA, Toyama-shi (JP); Gun SON, Toyama-shi (JP); Takahiro KISHIOKA, Toyama-shi (JP)

(73) Assignee: NISSAN CHEMICAL CORPORATION, Tokyo (JP)

(21) Appl. No.: 18/714,440

(22) PCT Filed: Oct. 7, 2022
(86) PCT No.: PCT/JP2022/037533

§ 371 Date: May 29, 2024

(30) Foreign Application Priority Data
Nov. 29, 2021 (JP) 2021-193025

(51) Int. Cl. C09D 163/00; C08G 59/32; C08G 59/40; C08G 59/68; C09D 7/20; G03F 7/09; G03F 7/11; G03F 7/16; H01L 21/02; H01L 21/027; H01L 21/306; H01L 21/308; H01L 21/311; H01L 21/3213

(52) U.S. Cl. CPC C09D 163/00; C08G 59/3245; C08G 59/4014; C08G 59/687; G03F 7/094; G03F 7/11; G03F 7/168; H01L 21/0275; H01L 21/3081; H01L 21/3086; H01L 21/31144; H01L 21/32139; C08G 2150/00; C09D 7/20; H01L 21/0206; H01L 21/02068; H01L 21/30612; H01L 21/31111; H01L 21/32134

(57) ABSTRACT

A composition for forming a protective film capable of forming a protective film excellent in resistance to a semiconductor wet etchant such as a basic hydrogen peroxide solution or an acidic hydrogen peroxide solution, which exhibits excellent resistance also to a resist solvent and can be effectively used also as a resist underlayer film-forming composition. A protective film-forming composition against a semiconductor wet etchant, including: (A) a compound represented by the following formula (A); and (B) a solvent.

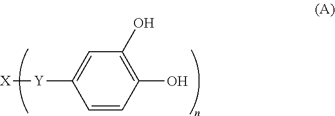

(In the formula (A), n represents an integer of 1 to 10, and when n is 2, X represents a sulfinyl group, a sulfonyl group, an ether group or a C₂₋₅₀ divalent organic group, and when n is an integer other than 2, X represents a C₂₋₅₀ n-valent organic group, Y represents —CH₂CH(OH)CH₂OC(═O)CH₂(CH₂)ₜ—, —CH₂CH(OH)CH₂OC(═O)C(CN)(═CH)—, and t represents an integer of 1 to 6.)

### TECHNICAL FIELD

The present invention relates to a composition for forming a protective film excellent in resistance particularly to a semiconductor wet etchant in a lithography process in semiconductor manufacturing. The present invention also relates to a protective film formed from the composition, a method for producing a resist-patterned substrate to which the protective film is applied, and a method for manufacturing a semiconductor device.

### BACKGROUND ART

In semiconductor manufacturing, a lithography process for forming a resist pattern having a desired shape by providing a resist underlayer film between a substrate and a resist film formed thereon is widely known. The substrate is processed after the resist pattern is formed, and dry etching is mainly used as the process, but wet etching may be used depending on the substrate type.

Patent Literature 1 discloses a resist underlayer film material having resistance to an alkaline hydrogen peroxide solution.

### CITATION LIST

### Patent Literature

Patent Literature 1: JP 2018-173520 A

### SUMMARY OF INVENTION

### Technical Problem

When a protective film for a semiconductor substrate is formed using a protective film-forming composition, and a base substrate is processed by wet etching through the protective film as an etching mask, the protective film is required to have a good mask function (that is, the masked portion can protect the substrate) for a semiconductor wet etchant.

Furthermore, there is also a demand for a protective film-forming composition that has good covering properties even for a so-called stepped substrate, has a small difference in film thickness after embedding, and can form a flat film.

Conventionally, in order to develop resistance to SC-1 (ammonia-hydrogen peroxide solution) which is a type of wet etching chemical, a method of applying a low molecular compound (for example, gallic acid) as an additive has been used, but there is a limit in solving the above problems. Therefore, a protective film-forming composition for forming a protective film exhibiting excellent resistance to a basic hydrogen peroxide solution such as SC-1 is desired.

In addition, since wet etching using a hydrogen peroxide solution is also performed, a protective film-forming composition for forming a protective film exhibiting excellent resistance to an acidic hydrogen peroxide solution is also desired.

Furthermore, the protective film used for the above purpose is expected to have a function as a so-called resist underlayer film, and is also desired to exhibit excellent resistance to a resist solvent.

The present invention has been made in view of the above circumstances, and an object of the present invention is to provide a composition for forming a protective film capable of forming a protective film excellent in resistance to a semiconductor wet etchant such as a basic hydrogen peroxide solution or an acidic hydrogen peroxide solution, which exhibits excellent resistance also to a resist solvent and can be effectively used also as a resist underlayer film-forming composition.

### Solution to Problem

As a result of intensive studies to solve the above problems, the present inventors have found that a film obtained from a composition for forming a protective film containing a compound represented by a specific structural formula having a catechol group exhibits excellent resistance to a semiconductor wet etchant, and have completed the present invention.

That is, the present invention includes the following aspects.
[1] A protective film-forming composition against a semiconductor wet etchant, containing:
   (A) a compound represented by the following formula (A); and
   (B) a solvent,

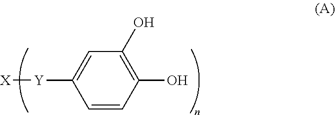

in the formula (A), n represents an integer of 1 to 10, and when n is 2, X represents a sulfinyl group, a sulfonyl group, an ether group or a C₂₋₅₀ divalent organic group, and when n is an integer other than 2, X represents a C₂₋₅₀ n-valent organic group, Y represents —CH₂CH(OH)CH₂OC(═O)CH₂(CH₂)ₜ—, —CH₂CH(OH)CH₂OC(═O)C(CN)(═CH)—, and t represents an integer of 1 to 6.
[2] The protective film-forming composition according to [1], wherein in the formula (A), when the X is a C₂₋₅₀ divalent organic group, the X is a divalent organic group represented by the following formula (A-1), and when the X is a C₂₋₅₀ n-valent organic group other than a C₂₋₅₀ divalent organic group, the X is an n-valent organic group represented by the following formula (A-2),

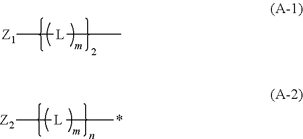

in the formula (A-1), Z₁ represents a C₁₋₆ alkylene group or a divalent organic group containing a ring selected from the group consisting of an aromatic ring optionally having a substituent, an aliphatic ring optionally having a substituent, and a heterocyclic ring optionally having a substituent, or a divalent organic group containing the ring and a C₁₋₆ alkylene group, m represents 0 or 1, and L represents —O— or —C(═O)—O—, and in the formula (A-2), Z₂ represents an n-valent organic group containing a ring selected from the group consisting of an aromatic ring optionally having a substituent, an aliphatic ring optionally having a substituent, and a heterocyclic ring optionally having a substituent, or an n-valent organic group containing the ring and a C₁₋₆ alkylene group, m represents 0 or 1, and L represents —O— or —C(═O)—O—.
[3] The protective film-forming composition according to [1] or [2], wherein the protective film-forming composition further includes at least one of (C) a crosslinking agent, (D) a crosslinking catalyst, and (E) a surfactant.
[4] The protective film-forming composition according to any one of [1] to [3], wherein the protective film-forming composition further contains (F) a compound or polymer containing a (meth)acryloyl group, a styrene group, a phenolic hydroxy group, an ether group, an epoxy group, or an oxetanyl group.
[5] The protective film-forming composition according to [4], wherein the protective film-forming composition further includes (G) a polymer having a repeating structural unit represented by the following formula (G):

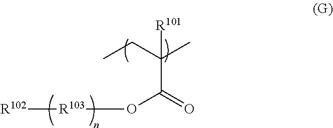

in the formula (G), R¹⁰¹ represents a hydrogen atom or a methyl group, R¹⁰² represents a group selected from the following formulas (g-1) to (g-3), a C₁₋₄ alkyl group optionally interrupted by oxygen, an aryl group optionally substituted, or a hydroxy group, R¹⁰³ represents a C₁₋₄ alkylene group, and n represents 0 or 1, and in the formulas (g-1) to (g-3), * represents a bond.

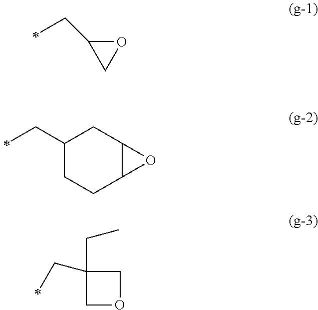

[6] The protective film-forming composition according to [4], further containing (J) a compound (J) or a polymer (J) containing a cyclic ether having a 3-membered ring structure or a 4-membered ring structure.
[7] A protective film against a semiconductor wet etchant, which is a baked product of a coating film including the protective film-forming composition according to any one of [1] to [6].
[8] A resist underlayer film-forming composition, containing:
   (A) a compound represented by the following formula (A); and
   (B) a solvent,

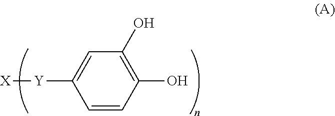

in the formula (A), n represents an integer of 1 to 10, and when n is 2, X represents a sulfinyl group, a sulfonyl group, an ether group or a C₂₋₅₀ divalent organic group, and when n is an integer other than 2, X represents a C₂₋₅₀ n-valent organic group, Y represents —CH₂CH(OH)CH₂OC(═O)CH₂(CH₂)ₜ—, —CH₂CH(OH)CH₂OC(═O)C(CN)(═CH)—, and t represents an integer of 1 to 6.
[9] The resist underlayer film-forming composition according to [8], wherein in the formula (A), when the X is a C₂₋₅₀ divalent organic group, the X is a divalent organic group represented by the following formula (A-1), and when the X is a C₂₋₅₀ n-valent organic group other than a C₂₋₅₀ divalent organic group, the X is an n-valent organic group represented by the following formula (A-2),

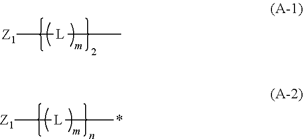

in the formula (A-1), Z₁ represents a C₁₋₆ alkylene group or a divalent organic group containing a ring selected from the group consisting of an aromatic ring optionally having a substituent, an aliphatic ring optionally having a substituent, and a heterocyclic ring optionally having a substituent, or a divalent organic group containing the ring and a C₁₋₆ alkylene group, m represents 0 or 1, and L represents —O— or —C(═O)—O—, and
in the formula (A-2), Z₂ represents an n-valent organic group containing a ring selected from the group consisting of an aromatic ring optionally having a substituent, an aliphatic ring optionally having a substituent, and a heterocyclic ring optionally having a substituent, or an n-valent organic group containing the ring and a C₁₋₆ alkylene group, m represents 0 or 1, and L represents —O— or —C(═O)—O—.
[10] A resist underlayer film, which is a baked product of a coating film including the resist underlayer film-forming composition according to [8] or [9].
[11] A method for producing a substrate with a protective film, including forming a protective film by applying the protective film-forming composition according to any one of [1] to [6] onto a stepped semiconductor substrate and baking the composition, wherein the substrate is used for producing a semiconductor.
[12] A method for producing a resist-patterned substrate, including applying the protective film-forming composition according to any one of [1] to [6] or the resist underlayer film-forming composition according to [8] or [9] onto a semiconductor substrate and baking the composition to form a protective film as a resist underlayer film; and forming a resist film on the protective film and then exposing and developing the composition to form a resist pattern, wherein the substrate is used for producing a semiconductor.
[13] A method for manufacturing a semiconductor device, including forming a protective film with the protective film-forming composition according to any one of [1] to [6]on a semiconductor substrate optionally having an inorganic film formed on a surface thereof, forming a resist pattern on the protective film, dry-etching the protective film through the resist pattern as a mask to expose a surface of the inorganic film or of the semiconductor substrate, and wet etching and cleaning the inorganic film or the semiconductor substrate with a semiconductor wet etchant through the dry-etched protective film as a mask.
[14] A method for manufacturing a semiconductor device, including forming a resist underlayer film with the resist underlayer film-forming composition according to [8] or [9]on a semiconductor substrate optionally having an inorganic film formed on a surface thereof, forming a resist pattern on the resist underlayer film, dry-etching the resist underlayer film through the resist pattern as a mask to expose a surface of the inorganic film or of the semiconductor substrate, and etching the inorganic film or the semiconductor substrate through the dry-etched resist underlayer film as a mask.

### Advantageous Effects of Invention

According to the present invention, it is possible to provide a composition for forming a protective film capable of forming a protective film excellent in resistance to a semiconductor wet etchant such as a basic hydrogen peroxide solution or an acidic hydrogen peroxide solution, which exhibits excellent resistance also to a resist solvent and can be effectively used also as a resist underlayer film-forming composition.

The protective film-forming composition of the present invention is required to have, for example, the following characteristics in a well-balanced manner in a lithography process in semiconductor manufacturing. (1) To have a good mask function for a wet etchant at the time of processing a base substrate, (2) to further reduce damage to a protective film or a resist underlayer film at the time of processing a substrate by a low dry etching rate, (3) to have excellent flatness of a stepped substrate, and (4) to have excellent embeddability in a fine trench pattern substrate. Moreover, the semiconductor substrate can be easily micromachined by having the performances (1) to (4) in a well-balanced manner.

### DESCRIPTION OF EMBODIMENTS

Hereinafter, the present invention will be described in detail. Note that the description of the constituent elements described below is an example for describing the present invention, and the present invention is not limited to these contents.

### (Protective Film-Forming Composition Against Semiconductor Wet Etchant)

The protective film-forming composition against the semiconductor wet etchant of the present invention contains:
(A) a compound represented by the following formula (A); and
(B) a solvent.

The protective film-forming composition of the present invention may contain at least one of (C) a crosslinking agent, (D) a crosslinking catalyst, and (E) a surfactant, in addition to the compound (A) represented by the following formula (A) and the solvent (B).

The protective film-forming composition of the present invention may also contain (F) a compound or polymer containing a (meth)acryloyl group, a styrene group, a phenolic hydroxy group, an ether group, an epoxy group, or an oxetanyl group, in addition to the compound (A) represented by the following formula (A) and the solvent (B).

### <(A) Compound Represented by Formula (A)>

The protective film-forming composition against the semiconductor wet etchant of the present invention contains the compound (A) represented by the following formula (A).

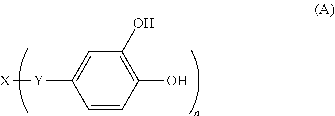

In the formula (A), n represents an integer of 1 to 10, and when n is 2, X represents a sulfinyl group, a sulfonyl group, an ether group or a C₂₋₅₀ divalent organic group, and when n is an integer other than 2, X represents a C₂₋₅₀ n-valent organic group. Y represents —CH₂CH(OH)CH₂OC(═O)CH₂(CH₂)ₜ—, —CH₂CH(OH)CH₂OC(═O)C(CN)(═CH)—, and t represents an integer of 1 to 6.

In the formula (A), when the X is a C₂₋₅₀ divalent organic group, the X is a divalent organic group represented by the following formula (A-1), and when the X is a C₂₋₅₀ n-valent organic group other than a C₂₋₅₀ divalent organic group, the X is an n-valent organic group represented by the following formula (A-2).

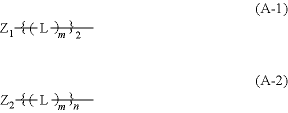

In the formula (A-1), Z₁ represents a C₁₋₆ alkylene group or a divalent organic group containing a ring selected from the group consisting of an aromatic ring optionally having a substituent, an aliphatic ring optionally having a substituent, and a heterocyclic ring optionally having a substituent, or a divalent organic group containing the ring and a C₁₋₆ alkylene group, m represents 0 or 1, and L represents —O— or —C(═O)—O—.

In the formula (A-2), Z₂ represents an n-valent organic group containing a ring selected from the group consisting of an aromatic ring optionally having a substituent, an aliphatic ring optionally having a substituent, and a heterocyclic ring optionally having a substituent, or an n-valent organic group containing the ring and a C₁₋₆ alkylene group, m represents 0 or 1, and L represents —O— or —C(═O)—O—.

In the formulas (A-1) and (A-2), the substituent referred in the aromatic ring optionally having a substituent, the aliphatic ring optionally having a substituent, or the heterocyclic ring optionally having a substituent represents a C₁₋₁₀ alkyl group optionally interrupted by an oxygen atom or a sulfur atom, a C₁₋₁₀ alkenyl group optionally interrupted by an oxygen atom or a sulfur atom, or a C₁₋₁₀ alkynyl group optionally interrupted by an oxygen atom or a sulfur atom. The alkyl group, the alkenyl group, and the alkynyl group may be linear or branched.

In the above formulas (A-1) and (A-2), the alkylene group refers to a divalent group derived by further removing one hydrogen atom from the alkyl group. The alkylene group may be linear or branched.

In the above formulas (A-1) and (A-2), specific examples of the aromatic ring include benzene, naphthalene, anthracene, acenaphthene, fluorene, triphenylene, phenalene, phenanthrene, indene, indane, indacene, pyrene, chrysene, perylene, naphthacene, pentacene, coronene, heptacene, benzo[a]anthracene, dibenzophenanthrene, and dibenzo[a,j]anthracene.

In the above formulas (A-1) and (A-2), specific examples of the heterocyclic ring include furan, thiophene, pyrrole, imidazole, pyran, pyridine, pyrimidine, pyrazine, pyrrolidine, piperidine, piperazine, morpholine, quinuclidine, indole, purine, thymine, quinoline, isoquinoline, chromene, thianthrene, phenothiazine, phenoxazine, xanthene, acridine, phenazine, carbazole, hydantoin, uracil, barbituric acid, triazine, and cyanuric acid. The heterocyclic ring may be a triazinetrione.

Examples of the C₁₋₁₀ alkyl group include a methyl group, an ethyl group, a n-propyl group, an i-propyl group, a cyclopropyl group, a n-butyl group, an i-butyl group, a s-butyl group, a t-butyl group, a cyclobutyl group, a 1-methyl-cyclopropyl group, a 2-methyl-cyclopropyl group, a n-pentyl group, a 1-methyl-n-butyl group, a 2-methyl-n-butyl group, a 3-methyl-n-butyl group, a 1,1-dimethyl-n-propyl group, a 1,2-dimethyl-n-propyl group, a 2,2-dimethyl-n-propyl group, a 1-ethyl-n-propyl group, a cyclopentyl group, a 1-methyl-cyclobutyl group, a 2-methyl-cyclobutyl group, a 3-methyl-cyclobutyl group, a 1,2-dimethyl-cyclopropyl group, a 2,3-dimethyl-cyclopropyl group, a 1-ethyl-cyclopropyl group, a 2-ethyl-cyclopropyl group, a n-hexyl group, a 1-methyl-n-pentyl group, a 2-methyl-n-pentyl group, a 3-methyl-n-pentyl group, a 4-methyl-n-pentyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, a 1-ethyl-n-butyl group, a 2-ethyl-n-butyl group, a 1,1,2-trimethyl-n-propyl group, a 1,2,2-trimethyl-n-propyl group, a 1-ethyl-1-methyl-n-propyl group, a 1-ethyl-2-methyl-n-propyl group, a cyclohexyl group, a 1-methyl-cyclopentyl group, a 2-methyl-cyclopentyl group, a 3-methyl-cyclopentyl group, a 1-ethyl-cyclobutyl group, a 2-ethyl-cyclobutyl group, a 3-ethyl-cyclobutyl group, a 1,2-dimethyl-cyclobutyl group, a 1,3-dimethyl-cyclobutyl group, a 2,2-dimethyl-cyclobutyl group, a 2,3-dimethyl-cyclobutyl group, a 2,4-dimethyl-cyclobutyl group, a 3,3-dimethyl-cyclobutyl group, a 1-n-propyl-cyclopropyl group, a 2-n-propyl-cyclopropyl group, a 1-i-propyl-cyclopropyl group, a 2-i-propyl-cyclopropyl group, a 1,2,2-trimethyl-cyclopropyl group, a 1,2,3-trimethyl-cyclopropyl group, a 2,2,3-trimethyl-cyclopropyl group, a 1-ethyl-2-methyl-cyclopropyl group, a 2-ethyl-1-methyl-cyclopropyl group, a 2-ethyl-2-methyl-cyclopropyl group, and a 2-ethyl-3-methyl-cyclopropyl group.

The phrase “optionally interrupted” means that any carbon-carbon atom in the alkyl group, the alkenyl group, or the alkynyl group is interrupted by a hetero atom (that is, an ether bond in the case of oxygen, and a sulfide bond in the case of sulfur).

The compound represented by the above formula (A) will be described below separately for the case where Y is —CH₂CH(OH)CH₂OC(═O)CH₂(CH₂)ₜ— and the case where Y is —CH₂CH(OH)CH₂OC(═O)C(CN)(═CH)—.

The case where Y is —CH₂CH(OH)CH₂OC(═O)CH₂(CH₂)ₜ— will be described in detail in the following section <<First Aspect>>, and the case where Y is —CH₂CH(OH)CH₂OC(═O)C(CN)(═CH)— will be described in detail in the following section <<Second Aspect>>.

### <<First Aspect>>

Among the compounds represented by the formula (A) according to the present invention, the compound in which Y is represented by —CH₂CH(OH)CH₂OC(═O)CH₂(CH₂)ₜ— is obtained, for example, by reacting an epoxy resin with a compound (a) represented by the following formula.

A reaction example for obtaining the compound represented by the formula (A) using, for example, triazinetrione as the epoxy resin and t in Y being 1 is shown below.

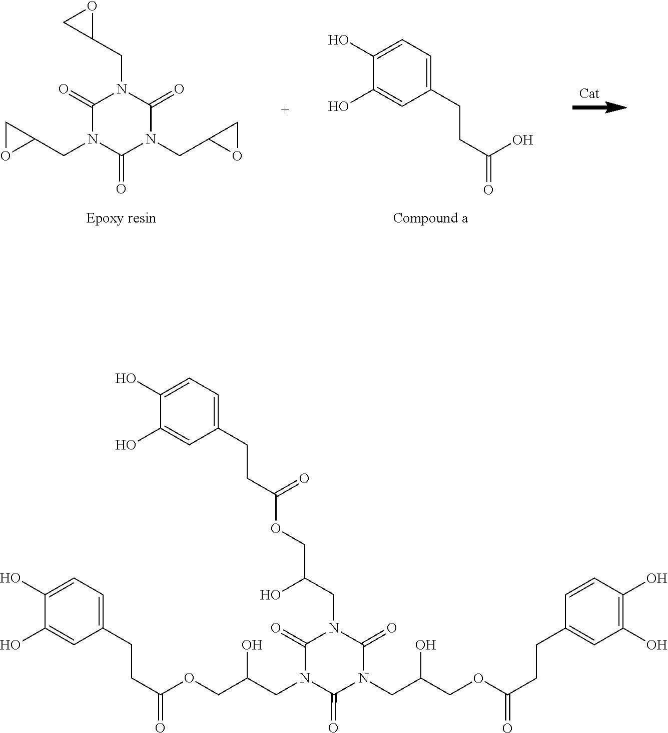 [SMILES: O=c1n(CC2CO2)c(=O)n(CC2CO2)c(=O)n1CC1CO1 ; O=C(O)CCc1ccc(O)c(O)c1 ; O=C(CCc1ccc(O)c(O)c1)OCC(O)Cn1c(=O)n(CC(O)COC(=O)CCc2ccc(O)c(O)c2)c(=O)n(CC(O)COC(=O)CCc2ccc(O)c(O)c2)c1=O]

Specific examples of the epoxy resin used for obtaining the compound represented by the above formula (A) include epoxy resins represented by the following.

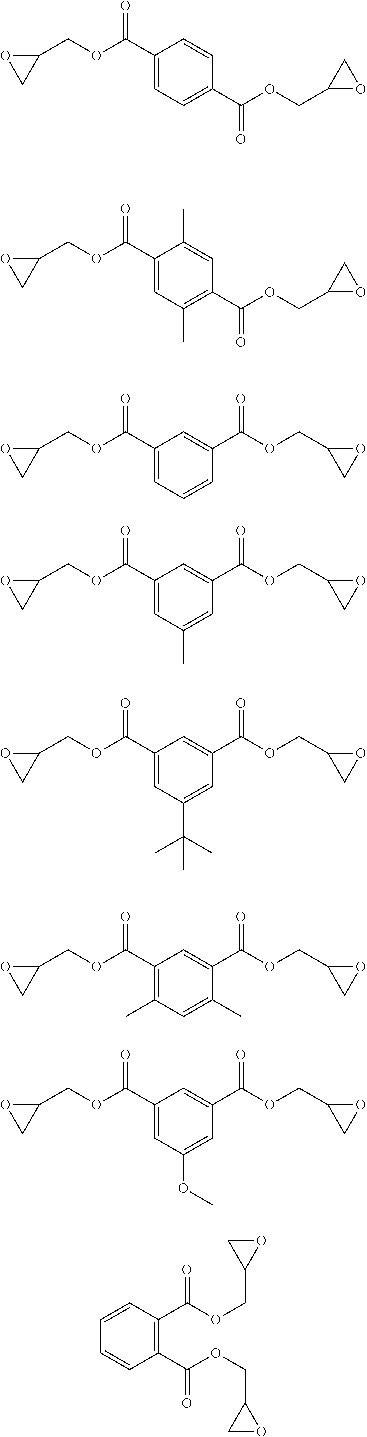

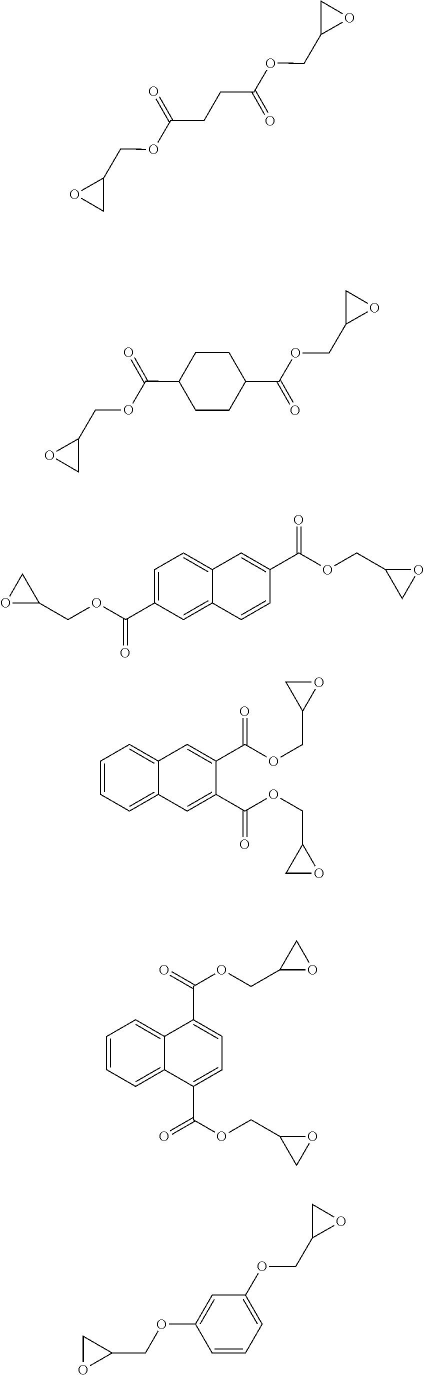

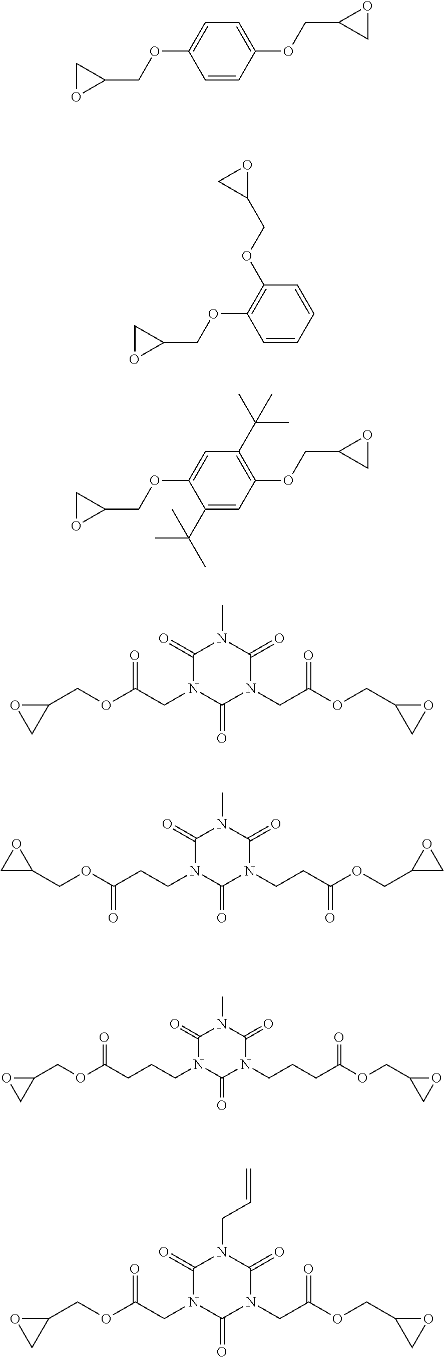

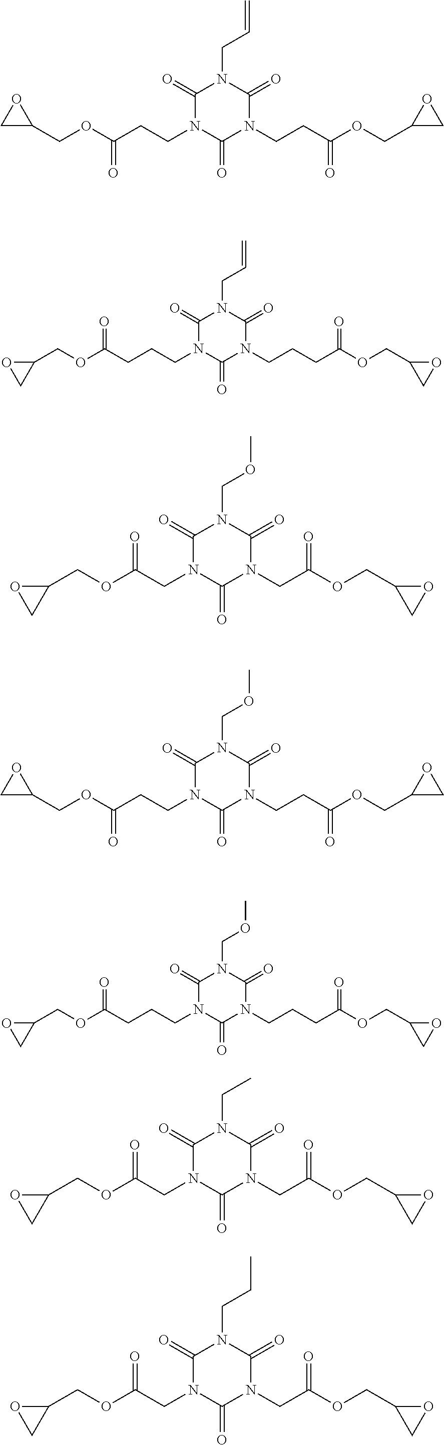

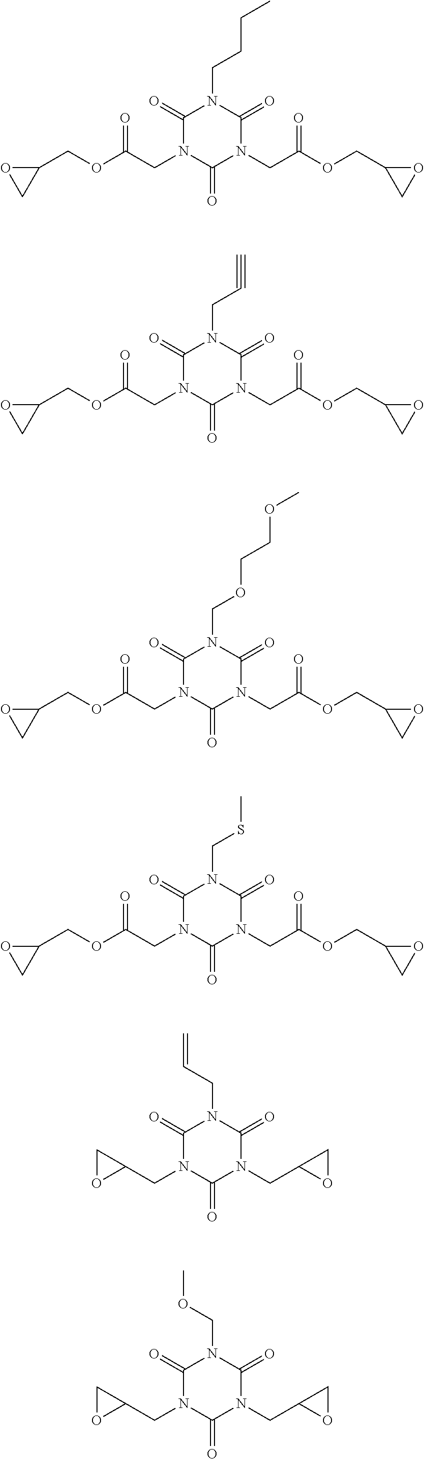 [SMILES: CCCCn1c(=O)n(CC(=O)OCC2CO2)c(=O)n(CC(=O)OCC2CO2)c1=O ; C#CCn1c(=O)n(CC(=O)OCC2CO2)c(=O)n(CC(=O)OCC2CO2)c1=O ; COCCOCn1c(=O)n(CC(=O)OCC2CO2)c(=O)n(CC(=O)OCC2CO2)c1=O ; CSCn1c(=O)n(CC(=O)OCC2CO2)c(=O)n(CC(=O)OCC2CO2)c1=O ; C=CCn1c(=O)n(CC2CO2)c(=O)n(CC2CO2)c1=O ; COCn1c(=O)n(CC2CO2)c(=O)n(CC2CO2)c1=O]

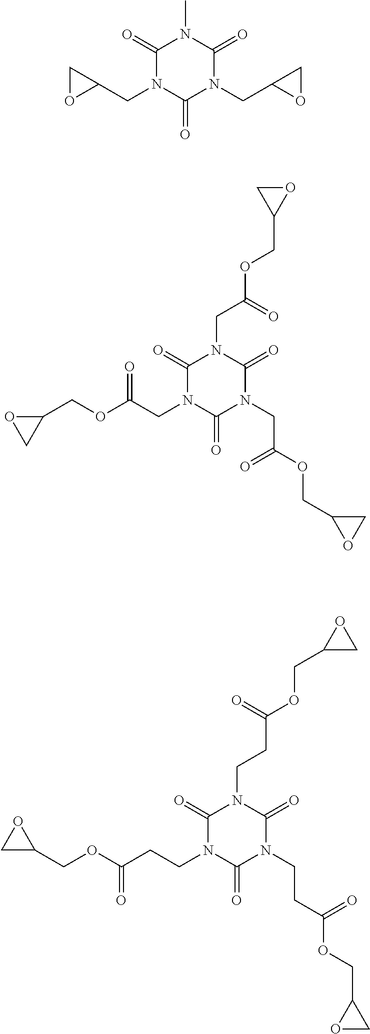 [SMILES: Cn1c(=O)n(CC2CO2)c(=O)n(CC2CO2)c1=O ; O=C(Cn1c(=O)n(CC(=O)OCC2CO2)c(=O)n(CC(=O)OCC2CO2)c1=O)OCC1CO1 ; O=C(CCn1c(=O)n(CCC(=O)OCC2CO2)c(=O)n(CCC(=O)OCC2CO2)c1=O)OCC1CO1]

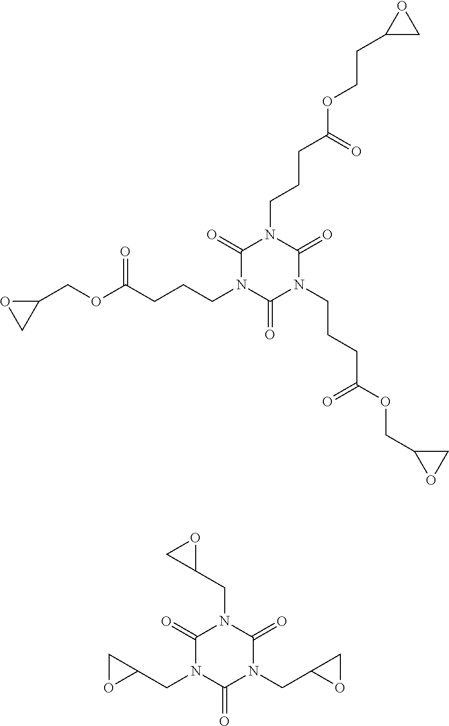 [SMILES: O=C(CCCn1c(=O)n(CCCC(=O)OCC2CO2)c(=O)n(CCCC(=O)OCC2CO2)c1=O)OCCC1CO1 ; O=c1n(CC2CO2)c(=O)n(CC2CO2)c(=O)n1CC1CO1]

### <<Second Aspect>>

Among the compounds represented by the formula (A) according to the present invention, a compound in which Y is represented by —CH₂CH(OH)CH₂OC(═O)C(CN)(═CH)— is obtained, for example, by reacting an epoxy resin with a compound (b1) represented by the following formula via an intermediate (b2) represented by the following formula.

A reaction example for obtaining the compound represented by the formula (A) using, for example, triazinetrione as the epoxy resin is shown below.

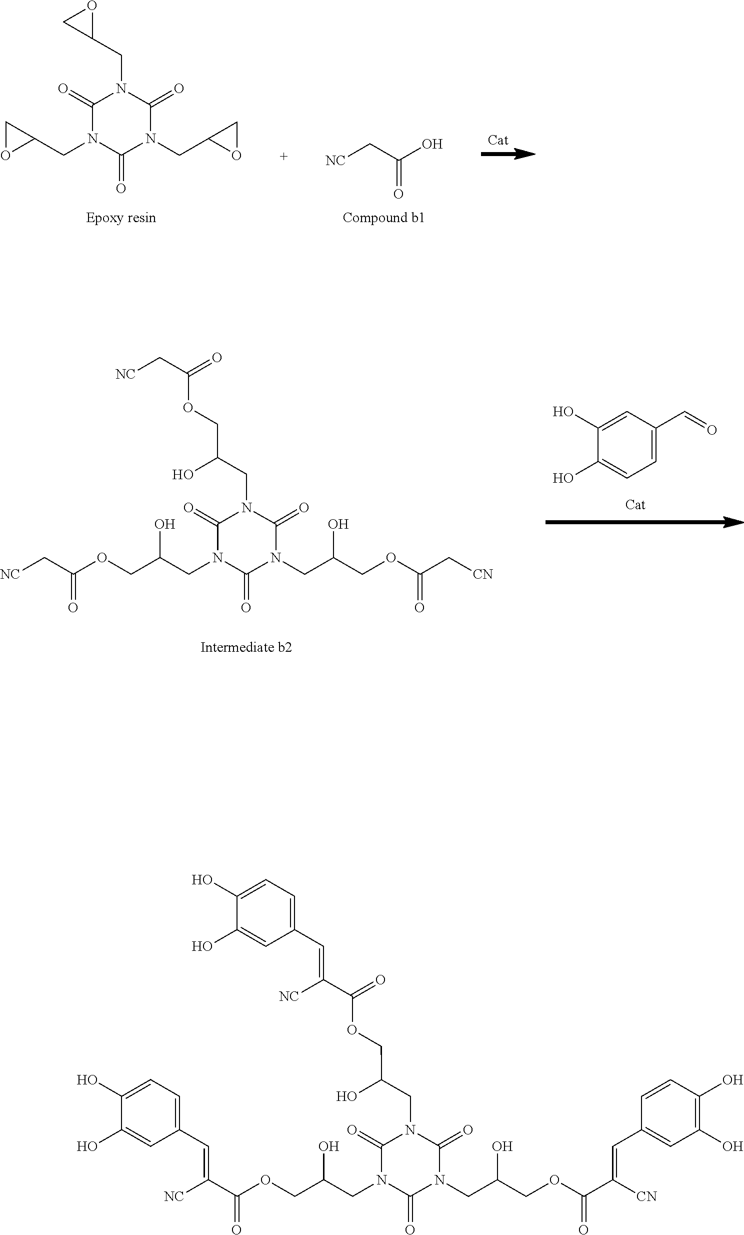 [SMILES: O=c1n(CC2CO2)c(=O)n(CC2CO2)c(=O)n1CC1CO1 ; N#CCC(=O)O ; N#CCC(=O)OCC(O)Cn1c(=O)n(CC(O)COC(=O)CC#N)c(=O)n(CC(O)COC(=O)CC#N)c1=O ; O=Cc1ccc(O)c(O)c1 ; N#C/C(=C\c1ccc(O)c(O)c1)C(=O)OCC(O)Cn1c(=O)n(CC(O)COC(=O)/C(C#N)=C/c2ccc(O)c(O)c2)c(=O)n(CC(O)COC(=O)/C(C#N)=C/c2ccc(O)c(O)c2)c1=O]

Specific examples of the epoxy resin used for obtaining the compound represented by the above formula (A) in the second aspect are as described in the description of the epoxy resin described in the section of <<First Aspect>>.

### <Solvent (B)>

The protective film-forming composition of the present invention can be prepared by dissolving the above-described components in a solvent, preferably an organic solvent, and is used in a uniform solution state.

The organic solvent of the protective film-forming composition according to the present invention can be used without particular limitation as long as it is an organic solvent capable of dissolving solid components such as the compound (A) and other optional solid components. In particular, since the protective film-forming composition according to the present invention is used in a uniform solution state, it is recommended to use an organic solvent generally used in a lithography process in combination in consideration of its application performance.

Examples of the organic solvent include ethylene glycol monomethyl ether, ethylene glycol monoethyl ether, methyl cellosolve acetate, ethyl cellosolve acetate, diethylene glycol monomethyl ether, diethylene glycol monoethyl ether, propylene glycol, propylene glycol monomethyl ether, propylene glycol monoethyl ether, propylene glycol monomethyl ether acetate, propylene glycol propyl ether acetate, toluene, xylene, methyl ethyl ketone, methyl isobutyl ketone, cyclopentanone, cyclohexanone, cycloheptanone, 4-methyl-2 pentanol, methyl 2-hydroxyisobutyrate, ethyl 2-hydroxyisobutyrate, ethyl ethoxyacetate, 2-hydroxyethyl acetate, methyl 3-methoxypropionate, ethyl 3-methoxypropionate, ethyl 3-ethoxypropionate, methyl 3-ethoxypropionate, methyl pyruvate, ethyl pyruvate, ethyl acetate, butyl acetate, ethyl lactate, butyl lactate, 2-heptanone, methoxy cyclopentane, anisole, y-butyrolactone, N-methylpyrrolidone, N,N-dimethylformamide, and N,N-dimethylacetamide. These solvents can be used singly or in combination of two or more thereof.

Among these solvents, propylene glycol monomethyl ether, propylene glycol monomethyl ether acetate, ethyl lactate, butyl lactate, cyclohexanone, and the like are preferable. In particular, propylene glycol monomethyl ether and propylene glycol monomethyl ether acetate are preferable.

The protective film-forming composition of the present invention may further contain at least one of (C) a crosslinking agent, (D) a crosslinking catalyst, and (E) a surfactant, in addition to the compound (A) represented by the following formula (A) and the solvent (B).

Furthermore, to the protective film-forming composition of the present invention, other components such as a light absorber, a rheology modifier, and an adhesion auxiliary can be added.

### <(C) Crosslinking Agent>

The protective film-forming composition of the present invention can contain a crosslinking agent component. Examples of the crosslinking agent include melamine crosslinking agents, substituted urea crosslinking agents, and crosslinking agents which are polymers thereof. Preferably, the crosslinking agent is a crosslinking agent having at least two crosslinking-forming substituents, and is a compound such as methoxymethylated glycoluril, butoxymethylated glycoluril, methoxymethylated melamine, butoxymethylated melamine, methoxymethylated benzoguanamine, butoxymethylated benzoguanamine, methoxymethylated urea, butoxymethylated urea, methoxymethylated thiourea, or methoxymethylated thiourea. In addition, condensates of these compounds can also be used.

In addition, as the crosslinking agent, a crosslinking agent having high heat resistance can be used. As the crosslinking agent having high heat resistance, a compound containing a crosslinking-forming substituent having an aromatic ring (for example, a benzene ring or a naphthalene ring) in the molecule can be used.

Examples of the compound include a compound having a partial structure of the following formula (5-1) and a polymer or oligomer having a repeating unit of the following formula (5-2).

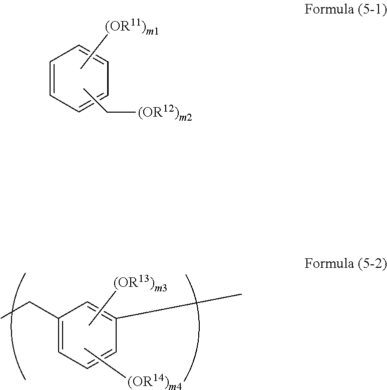

The R¹¹, R¹², R¹³, and R¹⁴ are each a hydrogen atom or a C₁₋₁₀ alkyl group, and examples described above can be used for these alkyl groups.

m1 is 1≤m1≤6−m2, m2 is 1≤m2≤5, m3 is 1≤m3≤4−m2, and m4 is 1≤m4≤3.

The compounds, polymers, and oligomers of the formula (5-1) and the formula (5-2) are exemplified below.

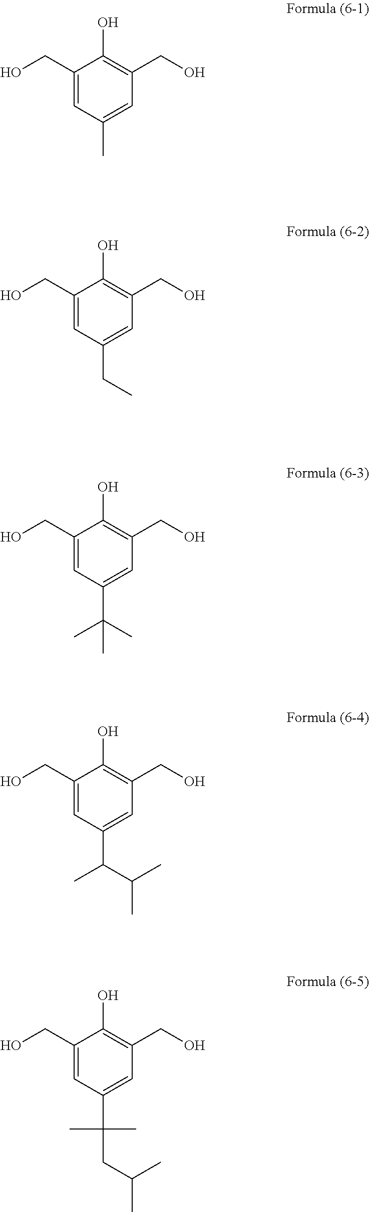 [SMILES: Cc1cc(CO)c(O)c(CO)c1 ; CCc1cc(CO)c(O)c(CO)c1 ; CC(C)(C)c1cc(CO)c(O)c(CO)c1 ; CC(C)C(C)c1cc(CO)c(O)c(CO)c1 ; CC(C)CC(C)(C)c1cc(CO)c(O)c(CO)c1]

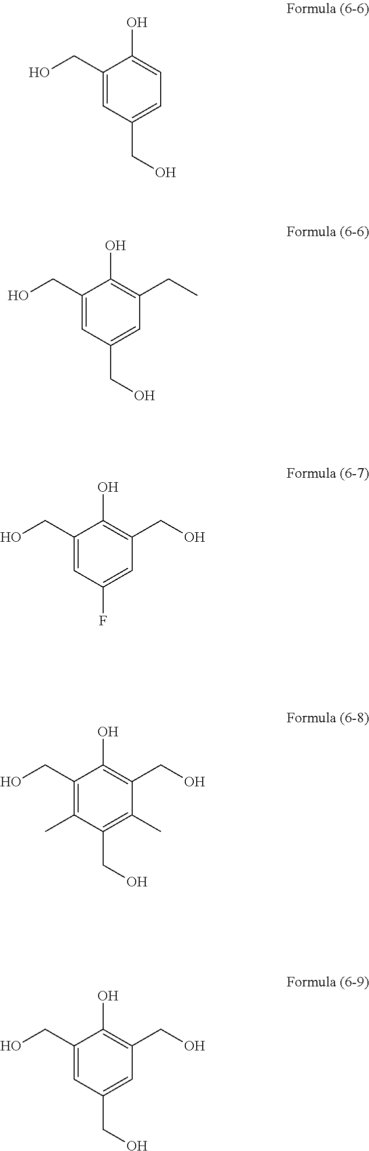

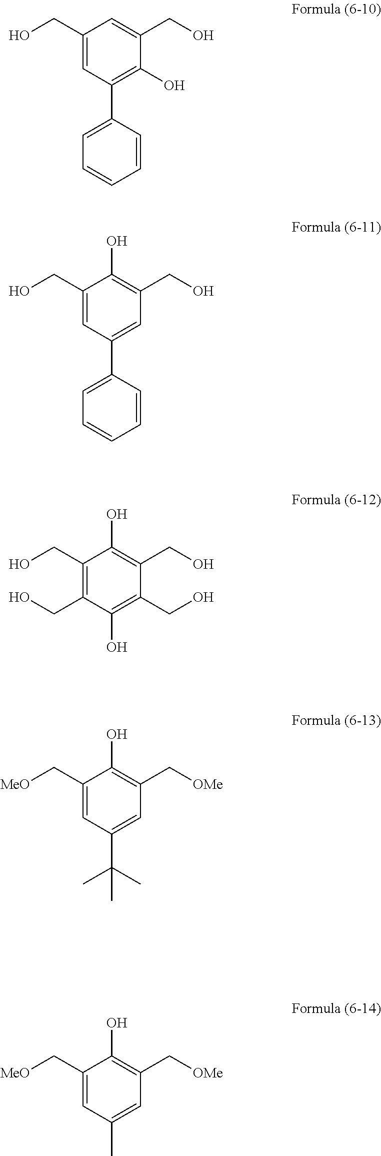 [SMILES: OCc1cc(CO)c(O)c(-c2ccccc2)c1 ; OCc1cc(-c2ccccc2)cc(CO)c1O ; OCc1c(O)c(CO)c(CO)c(O)c1CO ; COCc1cc(C(C)(C)C)cc(COC)c1O ; COCc1cc(C)cc(COC)c1O]

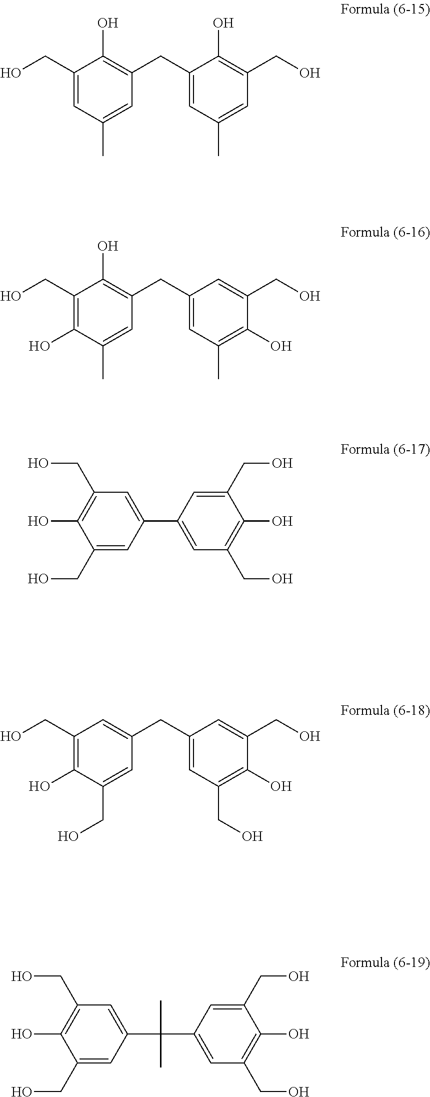 [SMILES: Cc1cc(CO)c(O)c(Cc2cc(C)cc(CO)c2O)c1 ; Cc1cc(Cc2cc(C)c(O)c(CO)c2O)cc(CO)c1O ; OCc1cc(-c2cc(CO)c(O)c(CO)c2)cc(CO)c1O ; OCc1cc(Cc2cc(CO)c(O)c(CO)c2)cc(CO)c1O ; CC(C)(c1cc(CO)c(O)c(CO)c1)c1cc(CO)c(O)c(CO)c1]

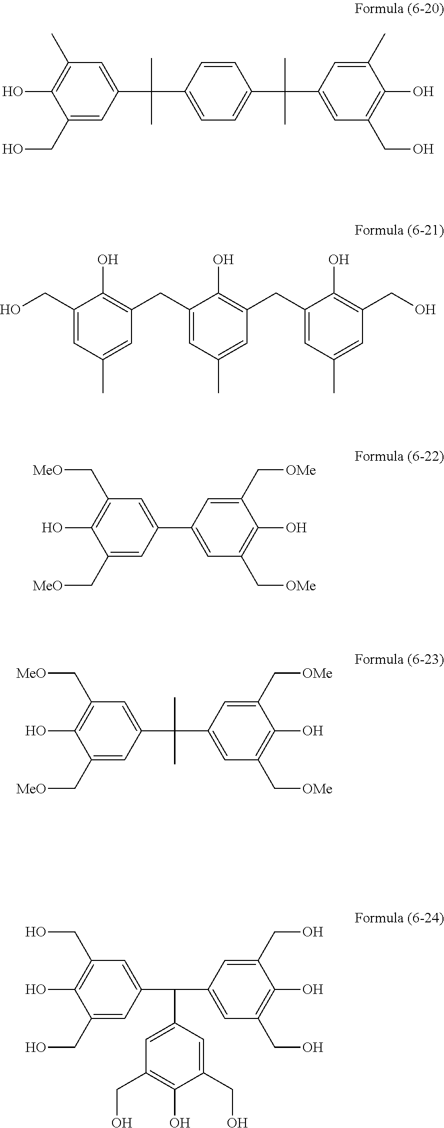 [SMILES: Cc1cc(C(C)(C)c2ccc(C(C)(C)c3cc(C)c(O)c(CO)c3)cc2)cc(CO)c1O ; Cc1cc(CO)c(O)c(Cc2cc(C)cc(Cc3cc(C)cc(CO)c3O)c2O)c1 ; COCc1cc(-c2cc(COC)c(O)c(COC)c2)cc(COC)c1O ; COCc1cc(C(C)(C)c2cc(COC)c(O)c(COC)c2)cc(COC)c1O ; OCc1cc(C(c2cc(CO)c(O)c(CO)c2)c2cc(CO)c(O)c(CO)c2)cc(CO)c1O]

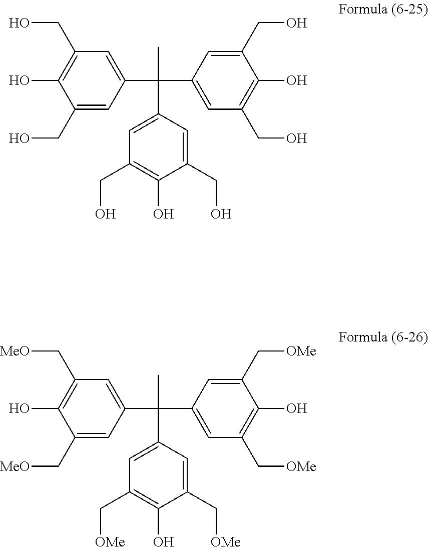 [SMILES: CC(c1cc(CO)c(O)c(CO)c1)(c1cc(CO)c(O)c(CO)c1)c1cc(CO)c(O)c(CO)c1 ; COCc1cc(C(C)(c2cc(COC)c(O)c(COC)c2)c2cc(COC)c(O)c(COC)c2)cc(COC)c1O]

The compound can be obtained as a product of ASAHI YUKIZAI CORPORATION or Honshu Chemical Industry Co., Ltd. For example, a compound of formula (6-22) among the above crosslinking agents can be obtained as TMOM-BP (trade name) manufactured by ASAHI YUKIZAI CORPORATION.

In the present invention, as in the TMOM-BP, a protective film exhibiting excellent resistance to a semiconductor wet etchant such as a basic hydrogen peroxide solution or an acidic hydrogen peroxide solution can be prepared by using a phenoplast crosslinking agent as compared with using other crosslinking agents (for example, an aminoplast crosslinking agent).

The amount of the crosslinking agent added varies depending on a coating solvent to be used, a base substrate to be used, required solution viscosity, required film shape, and the like, but is 0.001 to 80 mass %, preferably 0.01 to 50 mass %, and more preferably 0.1 to 40 mass % with respect to the total solid content of the protective film-forming composition. Although these crosslinking agents may cause a crosslinking reaction by self-condensation, they may cause a crosslinking reaction with a crosslinkable substituent in the polymer of the present invention described above when it is present.

### <(D) Crosslinking Catalyst>

The protective film-forming composition of the present invention can contain, as an optional component, a crosslinking catalyst for accelerating the crosslinking reaction. As the crosslinking catalyst, a compound that generates an acid or a base by heat can be used in addition to an acidic compound and a basic compound, but a crosslinking acid catalyst is preferable. As the acidic compound, a sulfonic acid compound or a carboxylic acid compound can be used, and as the compound that generates an acid by heat, a thermal acid generator can be used.

Examples of the sulfonic acid compound or carboxylic acid compound include p-toluenesulfonic acid, trifluoromethanesulfonic acid, pyridinium trifluoromethanesulfonate, pyridinium-p-toluenesulfonate, salicylic acid, camphorsulfonic acid, 5-sulfosalicylic acid, 4-chlorobenzenesulfonic acid, 4-hydroxybenzenesulfonic acid, pyridinium-4-hydroxybenzenesulfonic acid, benzenedisulfonic acid, 1-naphthalenesulfonic acid, 4-nitrobenzenesulfonic acid, citric acid, benzoic acid, and hydroxybenzoic acid.

Examples of the thermal acid generator include K-PURE [registered trademark] CXC-1612, CXC-1614, TAG-2172, TAG-2179, TAG-2678, and TAG2689 (all manufactured by King Industries), and SI-45, SI-60, SI-80, SI-100, SI-110, and SI-150 (all manufactured by Sanshin Chemical Industry Co., Ltd.).

Such crosslinking catalysts can be used singly or in combination of two or more thereof.

Also, as the basic compound, an amine compound or an ammonium hydroxide compound can be used, and as the compound that generates a base by heat, urea can be used.

Examples of the amine compound include tertiary amines such as triethanolamine, tributanolamine, trimethylamine, triethylamine, tri-n-propylamine, triisopropylamine, tri-n-butylamine, tri-tert-butylamine, tri-n-octylamine, triisopropanolamine, phenyldiethanolamine, stearyldiethanolamine, and diazabicyclooctane, and aromatic amines such as pyridine and 4-dimethylaminopyridine. Examples of the amine compound also include primary amines such as benzylamine and n-butylamine, and secondary amines such as diethylamine and di-n-butylamine. These amine compounds can be used singly or in combination of two or more thereof.

Examples of the ammonium hydroxide compound include tetramethylammonium hydroxide, tetraethylammonium hydroxide, tetrapropylammonium hydroxide, tetrabutylammonium hydroxide, benzyltrimethylammonium hydroxide, benzyltriethylammonium hydroxide, cetyltrimethylammonium hydroxide, phenyltrimethylammonium hydroxide, and phenyltriethylammonium hydroxide.

In addition, as the compound that generates a base by heat, for example, a compound having a thermolabile group such as an amide group, a urethane group, or an aziridine group and generating an amine by heating can be used. In addition, urea, benzyltrimethylammonium chloride, benzyltriethylammonium chloride, benzyldimethylphenylammonium chloride, benzyldodecyldimethylammonium chloride, benzyltributylammonium chloride, and choline chloride are also examples of compounds that generate a base by heat.

In the present invention, as in the case of using a crosslinked acid catalyst having a strong acid strength so as to generate a superacid like the trifluoromethanesulfonic acid, the degree of crosslinking increases, and the film strength of the protective film is increased. Therefore, a protective film exhibiting excellent resistance to a semiconductor wet etchant such as a basic hydrogen peroxide solution or an acidic hydrogen peroxide solution can be prepared.

When the protective film-forming composition contains a crosslinking catalyst, the content thereof is 0.0001 to 20 mass %, preferably 0.01 to 15 mass %, and more preferably 0.1 to 10 mass % with respect to the total solid content of the protective film-forming composition.

### <(E) Surfactant>

The protective film-forming composition of the present invention can contain, as an optional component, a surfactant for improving the coating property to a semiconductor substrate.

Examples of the surfactant include nonionic surfactants such as polyoxyethylene alkyl ethers such as polyoxyethylene lauryl ether, polyoxyethylene stearyl ether, polyoxyethylene cetyl ether and polyoxyethylene oleyl ether, polyoxyethylene alkylaryl ethers such as polyoxyethylene octylphenyl ether and polyoxyethylene nonylphenyl ether, polyoxyethylene/polyoxypropylene block copolymers, sorbitan fatty acid esters such as sorbitan monolaurate, sorbitan monopalmitate, sorbitan monostearate, sorbitan monooleate, sorbitan trioleate and sorbitan tristearate, and polyoxyethylene sorbitan fatty acid esters such as polyoxyethylene sorbitan monolaurate, polyoxyethylene sorbitan monopalmitate, polyoxyethylene sorbitan monostearate, polyoxyethylene sorbitan trioleate and polyoxyethylene sorbitan tristearate, fluorosurfactants such as EFTOP [registered trademark] EF301, EF303 and EF352 (manufactured by Mitsubishi Materials Electronic Chemicals Co., Ltd.), MEGAFACE [registered trademark] F171, F173, R-30, R-30N, R-40 and R-40-LM (manufactured by DIC CORPORATION), Fluorad FC430 and FC431 (manufactured by Sumitomo 3M Limited), AsahiGuard [registered trademark]AG710, and Surflon [registered trademark] S-382, SC101, SC102, SC103, SC104, SC105 and SC106 (manufactured by AGC Inc.), and organosiloxane polymer KP341 (manufactured by Shin-Etsu Chemical Co., Ltd.)

These surfactants can be used singly or in combination of two or more thereof.

When the protective film-forming composition contains a surfactant, the content thereof is 0.0001 to 10 mass %, preferably 0.01 to 5 mass % with respect to the total solid content of the protective film-forming composition.

### <Other Components>

To the protective film-forming composition of the present invention, a light absorber, a rheology modifier, an adhesion auxiliary, and the like can be added. The rheology modifier is effective for improving fluidity of the protective film-forming composition. The adhesion auxiliary is effective for improving adhesion between a semiconductor substrate or a resist and an underlayer film.

As the light absorber, commercially available light absorbers described in “Kougyouyou Shikiso no Gijutsu to Shijou (Technology and Market of Industrial Dyes)” (CMC Publishing Co., Ltd.) and “Senryou Binran (Dye Handbook)” (edited by The Society of Synthetic Organic Chemistry, Japan), such as, for example, C.I. Disperse Yellow 1, 3, 4, 5, 7, 8, 13, 23, 31, 49, 50, 51, 54, 60, 64, 66, 68, 79, 82, 88, 90, 93, 102, 114 and 124; C.I. Disperse Orange 1, 5, 13, 25, 29, 30, 31, 44, 57, 72 and 73; C.I. Disperse Red 1, 5, 7, 13, 17, 19, 43, 50, 54, 58, 65, 72, 73, 88, 117, 137, 143, 199 and 210; C.I. Disperse Violet 43; C.I. Disperse Blue 96; C.I. Fluorescent Brightening Agents 112, 135 and 163; C.I. Solvent Orange 2 and 45; C.I. Solvent Red 1, 3, 8, 23, 24, 25, 27 and 49; C.I. Pigment Green 10; C.I. Pigment Brown 2, and the like can be suitably used.

The light absorber is usually blended in a ratio of 10 mass % or less, preferably 5 mass % or less with respect to the total solid content of the protective film-forming composition.

The rheology modifier is added mainly for the purpose of improving the fluidity of the protective film-forming composition, and particularly in the baking step, for the purpose of improving the film thickness uniformity of the resist underlayer film and improving filling property of the protective film-forming composition into holes. Specific examples thereof include phthalic acid derivatives such as dimethyl phthalate, diethyl phthalate, diisobutyl phthalate, dihexyl phthalate, and butyl isodecyl phthalate; adipic acid derivatives such as di-n-butyl adipate, diisobutyl adipate, diisooctyl adipate, and octyl decyl adipate; maleic acid derivatives such as di-n-butyl maleate, diethyl maleate, and dinonyl maleate; oleic acid derivatives such as methyl oleate, butyl oleate, and tetrahydrofurfuryl oleate; and stearic acid derivatives such as n-butyl stearate and glyceryl stearate.

These rheology modifiers are usually blended in a ratio of less than 30 mass % with respect to the total solid content of the protective film-forming composition.

The adhesion auxiliary is added mainly for the purpose of improving adhesion between the substrate or the resist and the protective film-forming composition and thus preventing peeling of the resist particular during development. Specific examples thereof include chlorosilanes such as trimethylchlorosilane, dimethylmethylolchlorosilane, methyldiphenylchlorosilane, and chloromethyldimethylchlorosilane; alkoxysilanes such as trimethylmethoxysilane, dimethyldiethoxysilane, methyldimethoxysilane, dimethylmethylolethoxysilane, diphenyldimethoxysilane, and phenyltriethoxysilane; silazanes such as hexamethyldisilazane, N,N′-bis(trimethylsilyl)urea, dimethyltrimethylsilylamine, and trimethylsilylimidazole; silanes such as methyloltrichlorosilane, γ-chloropropyltrimethoxysilane, γ-aminopropyltriethoxysilane, and γ-glycidoxypropyltrimethoxysilane; heterocyclic compounds such as benzotriazole, benzimidazole, indazole, imidazole, 2-mercaptobenzimidazole, 2-mercaptobenzothiazole, 2-mercaptobenzoxazole, urazole, thiouracil, mercaptoimidazole, and mercaptopyrimidine; and urea such as 1,1-dimethylurea and 1,3-dimethylurea, or thiourea compounds.

These adhesion auxiliaries are usually blended in a ratio of less than 5% by mass, preferably less than 2% by mass with respect to the total solid content of the protective film-forming composition.

The protective film-forming composition of the present invention may further contain a compound or polymer containing a (meth)acryloyl group, a styrene group, a phenolic hydroxy group, an ether group, an epoxy group, or an oxetanyl group (hereinafter, also referred to as (F) other compound or polymer), in addition to the compound (A) represented by the following formula (A) and the solvent (B).

Here, the (meth)acryloyl group means an acryloyl group or a methacryloyl group.

### <(F) Other Compound or Polymer>

The protective film-forming composition of the present invention may contain, as (F) other compound or polymer, a compound or polymer containing a (meth)acryloyl group, a styrene group, a phenolic hydroxy group, an ether group, an epoxy group, or an oxetanyl group.

The solid content of the protective film-forming composition according to the present invention is usually 0.1 to 70 mass %, and preferably 0.1 to 60 mass %. The solid content is the content ratio of all components excluding the solvent from the protective film-forming composition. The content ratio of the compound (A) represented by the formula (A) in the solid content is preferably 1 to 100 mass %, more preferably 1 to 99.9 mass %, still more preferably 50 to 99.9 mass %, still more preferably 50 to 95 mass %, and particularly preferably 50 to 90 mass %.

In the protective film-forming composition of the present invention, even in an aspect in which a relatively small amount of the compound (A) represented by the formula (A) is added as an additive to the other compound or polymer (F), the effect of resistance to a semiconductor wet etchant such as a basic hydrogen peroxide solution or an acidic hydrogen peroxide solution and the effect of resistance to a resist solvent are exhibited (see the results of the following Examples).

When the compound (A) represented by the formula (A) is added to the other compound or polymer (F), the compound (A) represented by the formula (A) may be added in an amount of 5 to 50 mass % with respect to the solid component in the protective film-forming composition.

Preferred embodiments of the other compound or polymer (F) include, for example, (G) a polymer having a repeating structural unit represented by the following formula (G), and (J) a compound (J) or a polymer (J) containing a cyclic ether having a 3-membered ring structure or a 4-membered ring structure. In some cases, a polymer corresponding to either of (G) and (J) above is present, but in the present invention, it is not necessary to strictly distinguish between (G) and (J), and any polymer corresponding to either of (G) and (J) can be used as a component to be contained in the protective film-forming composition of the present invention.

### <(G) Polymer Having Repeating Structural Unit Represented by Following Formula (G)>

The protective film-forming composition of the present invention may contain (G) a polymer having a repeating structural unit represented by the following formula (G), in addition to the compound represented by the above formula (A) and the solvent (B).

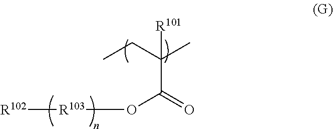

in the formula (G), R¹⁰¹ represents a hydrogen atom or a methyl group, R¹⁰² represents a group selected from the following formulas (g-1) to (g-3), a C₁₋₁₂ alkyl group optionally interrupted by oxygen, an aryl group optionally substituted, or a hydroxy group, R¹⁰³ represents a C₁₋₄ alkylene group, and n represents 0 or 1, and in the formulas (g-1) to (g-3), * represents a bond.

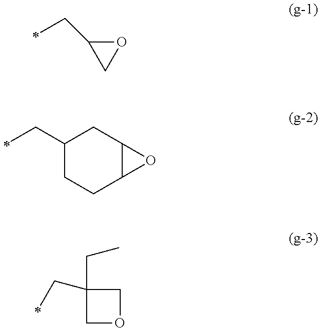

In the formula (G), examples of the substituent referred in the optionally substituted aryl group include an amino group and a hydroxy group.

Examples of the aryl group include a phenyl group, a naphthyl group, a biphenyl group, and an anthryl group.

The alkyl group may be linear, branched, or cyclic.

The phrase “optionally interrupted” means that any carbon-carbon atom in the alkyl group is interrupted by a hetero atom (that is, an ether bond in the case of oxygen).

The alkylene group refers to a divalent group derived by further removing one hydrogen atom from the alkyl group.

### <(J) Compound or Polymer>

The protective film-forming composition of the present invention may contain (J) a compound (J) or a polymer (J) containing a cyclic ether having a 3-membered ring structure or a 4-membered ring structure, in addition to the compound represented by the above formula (A) and the solvent (B).

Here, examples of the cyclic ether having a 3-membered ring structure include an epoxy group. Examples of the cyclic ether having a 4-membered ring structure include an oxetanyl group.

More preferred embodiments of the compound or polymer (J) include a compound represented by the following third aspect, and a polymer represented by the fourth aspect.

### <<Third Aspect>>

Examples of the compound (J) used in the present invention include the following compounds.

The compound (hereinafter, also referred to as a compound in the third aspect) is a compound having no repeating structural unit, and
contains a terminal group (J1), a polyvalent group (J2), and a linking group (J3), in which
the terminal group (J1) is bonded only to the linking group (J3),
the polyvalent group (J2) is bonded only to the linking group (J3), and
the linking group (J3) is bonded to the terminal group (J1) on the one hand and to the polyvalent group (J2) on the other hand and optionally bonded to another linking group (J3),
the terminal group (J1) has any of the structures of the following formula (I):

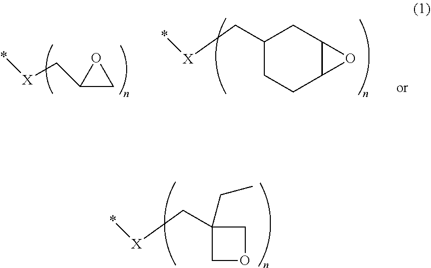

in the formula (I), * represents a binding site with a linking group (J3),
X represents an ether bond, an ester bond or a nitrogen atom, n=1 when X is an ether bond or an ester bond, and n=2 when X is a nitrogen atom,
the polyvalent group (J2) is
a divalent to tetravalent group selected from the group consisting of:
—O—,
aliphatic hydrocarbon groups,
combinations of an aromatic hydrocarbon group having less than 10 carbon atoms and an aliphatic hydrocarbon group, and
combinations of an aromatic hydrocarbon group having 10 or more carbon atoms and —O—, and
the linking group (J3) represents an aromatic hydrocarbon group.

The phrase “having no repeating structural unit” means to exclude a so-called polymer having a repeating structural unit, such as polyolefin, polyester, polyamide, or poly(meth)acrylate. The weight average molecular weight of the compound (J) is preferably 300 or more and 1,500 or less.

The “bond” among the terminal group (J1), the polyvalent group (J2), and the linking group (J3) means a chemical bond, and usually means a covalent bond, but does not preclude an ionic bond.

The polyvalent group (J2) is a divalent to tetravalent group.

Therefore, the aliphatic hydrocarbon group in the definition of the polyvalent group (J2) is a divalent to tetravalent aliphatic hydrocarbon group.

By way of non-limiting example, the divalent aliphatic hydrocarbon groups are exemplified by alkylene groups of a methylene group, an ethylene group, a n-propylene group, an isopropylene group, a cyclopropylene group, a n-butylene group, an isobutylene group, a s-butylene group, a t-butylene group, a cyclobutylene group, a 1-methyl-cyclopropylene group, a 2-methyl-cyclopropylene group, a n-pentylene group, a 1-methyl-n-butylene group, a 2-methyl-n-butylene group, a 3-methyl-n-butylene group, a 1,1-dimethyl-n-propylene group, a 1,2-dimethyl-n-propylene group, a 2,2-dimethyl-n-propylene, a 1-ethyl-n-propylene group, a cyclopentylene group, a 1-methyl-cyclobutylene group, a 2-methyl-cyclobutylene group, a 3-methyl-cyclobutylene group, a 1,2-dimethyl-cyclopropylene group, a 2,3-dimethyl-cyclopropylene group, a 1-ethyl-cyclopropylene group, a 2-ethyl-cyclopropylene group, a n-hexylene group, a 1-methyl-n-pentylene group, a 2-methyl-n-pentylene group, a 3-methyl-n-pentylene group, a 4-methyl-n-pentylene group, a 1,1-dimethyl-n-butylene group, a 1,2-dimethyl-n-butylene group, a 1,3-dimethyl-n-butylene group, a 2,2-dimethyl-n-butylene group, a 2,3-dimethyl-n-butylene group, a 3,3-dimethyl-n-butylene group, a 1-ethyl-n-butylene group, a 2-ethyl-n-butylene group, a 1,1,2-trimethyl-n-propylene group, a 1,2,2-trimethyl-n-propylene group, a 1-ethyl-1-methyl-n-propylene group, a 1-ethyl-2-methyl-n-propylene group, a cyclohexylene group, a 1-methyl-cyclopentylene group, a 2-methyl-cyclopentylene group, a 3-methyl-cyclopentylene group, a 1-ethyl-cyclobutylene group, a 2-ethyl-cyclobutylene group, a 3-ethyl-cyclobutylene group, a 1,2-dimethyl-cyclobutylene group, a 1,3-dimethyl-cyclobutylene group, a 2,2-dimethyl-cyclobutylene group, a 2,3-dimethyl-cyclobutylene group, a 2,4-dimethyl-cyclobutylene group, a 3,3-dimethyl-cyclobutylene group, a 1-n-propyl-cyclopropylene group, a 2-n-propyl-cyclopropylene group, a 1-isopropyl-cyclopropylene group, a 2-isopropyl-cyclopropylene group, a 1,2,2-trimethyl-cyclopropylene group, a 1,2,3-trimethyl-cyclopropylene group, a 2,2,3-trimethyl-cyclopropylene group, a 1-ethyl-2-methyl-cyclopropylene group, a 2-ethyl-1-methyl-cyclopropylene group, a 2-ethyl-2-methyl-cyclopropylene group, a 2-ethyl-3-methyl-cyclopropylene group, a n-heptylene group, a n-octylene group, a n-nonylene group, and a n-decanylene group.

Hydrogens at arbitrary sites are removed from these groups and converted into bonds, thereby deriving trivalent and tetravalent groups.

Examples of the aromatic hydrocarbon group having less than 10 carbon atoms in the definition of the polyvalent group (J2) include benzene, toluene, xylene, mesitylene, cumene, styrene, and indene.

Examples of the aliphatic hydrocarbon group combined with the aromatic hydrocarbon group having less than 10 carbon atoms include, in addition to the alkylene groups, alkyl groups such as a methyl group, an ethyl group, a n-propyl group, an i-propyl group, a cyclopropyl group, a n-butyl group, an i-butyl group, a s-butyl group, a t-butyl group, a cyclobutyl group, a 1-methyl-cyclopropyl group, a 2-methyl-cyclopropyl group, a n-pentyl group, a 1-methyl-n-butyl group, a 2-methyl-n-butyl group, a 3-methyl-n-butyl group, a 1,1-dimethyl-n-propyl group, a 1,2-dimethyl-n-propyl group, a 2,2-dimethyl-n-propyl group, a 1-ethyl-n-propyl group, a cyclopentyl group, a 1-methyl-cyclobutyl group, a 2-methyl-cyclobutyl group, a 3-methyl-cyclobutyl group, a 1,2-dimethyl-cyclopropyl group, a 2,3-dimethyl-cyclopropyl group, a 1-ethyl-cyclopropyl group, a 2-ethyl-cyclopropyl group, a n-hexyl group, a 1-methyl-n-pentyl group, a 2-methyl-n-pentyl group, a 3-methyl-n-pentyl group, a 4-methyl-n-pentyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, a 1-ethyl-n-butyl group, a 2-ethyl-n-butyl group, a 1,1,2-trimethyl-n-propyl group, a 1,2,2-trimethyl-n-propyl group, a 1-ethyl-1-methyl-n-propyl group, a 1-ethyl-2-methyl-n-propyl group, a cyclohexyl group, a 1-methyl-cyclopentyl group, a 2-methyl-cyclopentyl group, a 3-methyl-cyclopentyl group, a 1-ethyl-cyclobutyl group, a 2-ethyl-cyclobutyl group, a 3-ethyl-cyclobutyl group, a 1,2-dimethyl-cyclobutyl group, a 1,3-dimethyl-cyclobutyl group, a 2,2-dimethyl-cyclobutyl group, a 2,3-dimethyl-cyclobutyl group, a 2,4-dimethyl-cyclobutyl group, a 3,3-dimethyl-cyclobutyl group, a 1-n-propyl-cyclopropyl group, a 2-n-propyl-cyclopropyl group, a 1-i-propyl-cyclopropyl group, a 2-i-propyl-cyclopropyl group, a 1,2,2-trimethyl-cyclopropyl group, a 1,2,3-trimethyl-cyclopropyl group, a 2,2,3-trimethyl-cyclopropyl group, a 1-ethyl-2-methyl-cyclopropyl group, a 2-ethyl-1-methyl-cyclopropyl group, a 2-ethyl-2-methyl-cyclopropyl group, a 2-ethyl-3-methyl-cyclopropyl group, and a decyl group.

Any of the aromatic hydrocarbon group having less than 10 carbon atoms and the aliphatic hydrocarbon group in the definition of the polyvalent group (J2) may be bonded to the linking group (J3).

Examples of the aromatic hydrocarbon group having 10 or more carbon atoms in the definition of the polyvalent group (J2) include naphthalene, azulene, anthracene, phenanthrene, naphthacene, triphenylene, pyrene, and chrysene.

The aromatic hydrocarbon group having 10 or more carbon atoms in the definition of the polyvalent group (J2) is preferably bonded to the linking group (J3) via —O—.

Examples of the aromatic hydrocarbon group in the definition of the linking group (J3) include the aromatic hydrocarbon group having less than 10 carbon atoms and the aromatic hydrocarbon group having 10 or more carbon atoms.

Preferably, the compound (J) has two or more linking groups (J3).

The compound in the third aspect is preferably represented by, for example, the following formula (II).

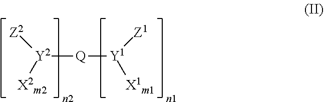

In the formula (II),

Z¹ and Z² are each independently

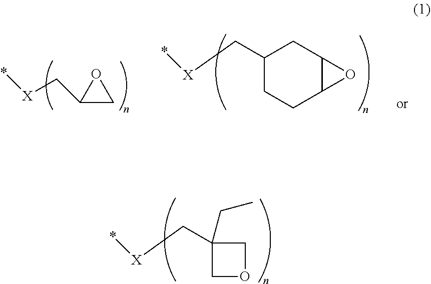

in the formula (I), * represents a binding site with Y¹ or Y²,
X represents an ether bond, an ester bond or a nitrogen atom, n=1 when X is an ether bond or an ester bond, and n=2 when X is a nitrogen atom, and
Y¹ and Y² each independently represent an aromatic hydrocarbon group,
X¹ and X² each independently represent —Y¹—Z¹ or —Y²—Z²,
n1 and n2 each independently represent an integer of 0 to 4, provided that any one of n1 and n2 is 1 or more,
m1 defined in (X¹)m1 represents 0 or 1,
m2 defined in (X²)m2 represents 0 or 1, and
Q represents a (n1+n2)-valent group selected from the group consisting of —O—, aliphatic hydrocarbon groups, combinations of an aromatic hydrocarbon group having less than 10 carbon atoms and an aliphatic hydrocarbon group, and combinations of an aromatic hydrocarbon group having 10 or more carbon atoms and —O—, and
Q is preferably a divalent to tetravalent group.

In the formula (II), Z¹ and Z² correspond to the terminal group (J1), Q corresponds to the polyvalent group (J2), Y¹ and Y² correspond to the linking group (J3), and the descriptions, examples, and the like thereof are as described above.

The compound in the third aspect preferably contains, for example, a partial structure represented by the following formula (III).

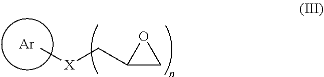

In the formula (III), Ar represents a benzene ring, a naphthalene ring, or an anthracene ring, and X represents an ether bond, an ester bond or a nitrogen atom, n=1 when X is an ether bond or an ester bond, and n=2 when X is a nitrogen atom.

### <<Fourth Aspect>>

Examples of the polymer (J) used in the present invention include the following polymers.

The polymer (hereinafter, also referred to as a polymer in the fourth aspect) is a polymer having a unit structure represented by the following formula (1-1):

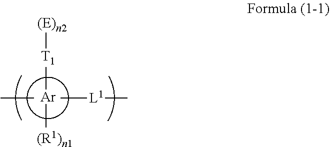

in the formula (1-1), Ar represents a benzene ring, a naphthalene ring, or an anthracene ring, R¹ represents a hydroxy group, a mercapto group which may be protected by a methyl group, an amino group which may be protected by a methyl group, a halogeno group, or a C₁₋₁₀ alkyl group which may be substituted with a hetero atom or substituted with a hydroxy group, n1 represents an integer of 0 to 3, L¹ represents a single bond or a C₁₋₁₀ alkylene group, n2 represents 1 or 2, E represents a group having an epoxy group or a group having an oxetanyl group, and T¹ represents a C₁₋₁₀ alkylene group optionally interrupted by a single bond, an ether bond, an ester bond, or an amide bond when n2=1, and T¹ represents a nitrogen atom or an amide bond when n2=2.

Examples of the C₁₋₁₀ alkyl group include a methyl group, an ethyl group, a n-propyl group, an i-propyl group, a cyclopropyl group, a n-butyl group, an i-butyl group, a s-butyl group, a t-butyl group, a cyclobutyl group, a 1-methyl-cyclopropyl group, a 2-methyl-cyclopropyl group, a n-pentyl group, a 1-methyl-n-butyl group, a 2-methyl-n-butyl group, a 3-methyl-n-butyl group, a 1,1-dimethyl-n-propyl group, a 1,2-dimethyl-n-propyl group, a 2,2-dimethyl-n-propyl group, a 1-ethyl-n-propyl group, a cyclopentyl group, a 1-methyl-cyclobutyl group, a 2-methyl-cyclobutyl group, a 3-methyl-cyclobutyl group, a 1,2-dimethyl-cyclopropyl group, a 2,3-dimethyl-cyclopropyl group, a 1-ethyl-cyclopropyl group, a 2-ethyl-cyclopropyl group, a n-hexyl group, a 1-methyl-n-pentyl group, a 2-methyl-n-pentyl group, a 3-methyl-n-pentyl group, a 4-methyl-n-pentyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, a 1-ethyl-n-butyl group, a 2-ethyl-n-butyl group, a 1,1,2-trimethyl-n-propyl group, a 1,2,2-trimethyl-n-propyl group, a 1-ethyl-1-methyl-n-propyl group, a 1-ethyl-2-methyl-n-propyl group, a cyclohexyl group, a 1-methyl-cyclopentyl group, a 2-methyl-cyclopentyl group, a 3-methyl-cyclopentyl group, a 1-ethyl-cyclobutyl group, a 2-ethyl-cyclobutyl group, a 3-ethyl-cyclobutyl group, a 1,2-dimethyl-cyclobutyl group, a 1,3-dimethyl-cyclobutyl group, a 2,2-dimethyl-cyclobutyl group, a 2,3-dimethyl-cyclobutyl group, a 2,4-dimethyl-cyclobutyl group, a 3,3-dimethyl-cyclobutyl group, a 1-n-propyl-cyclopropyl group, a 2-n-propyl-cyclopropyl group, a 1-i-propyl-cyclopropyl group, a 2-i-propyl-cyclopropyl group, a 1,2,2-trimethyl-cyclopropyl group, a 1,2,3-trimethyl-cyclopropyl group, a 2,2,3-trimethyl-cyclopropyl group, a 1-ethyl-2-methyl-cyclopropyl group, a 2-ethyl-1-methyl-cyclopropyl group, a 2-ethyl-2-methyl-cyclopropyl group, a 2-ethyl-3-methyl-cyclopropyl group, a decyl group, a methoxy group, an ethoxy group, a methoxymethyl group, an ethoxymethyl group, a methoxyethyl group, an ethoxyethyl group, a hydroxymethyl group, a 1-hydroxyethyl group, a 2-hydroxyethyl group, a methylamino group, a dimethylamino group, a diethylamino group, an aminomethyl group, a 1-aminoethyl group, a 2-aminoethyl group, a methylthio group, an ethylthio group, a mercaptomethyl group, a 1-mercaptoethyl group, and a 2-mercaptoethyl group.

Examples of the C₁₋₁₀ alkylene group include a methylene group, an ethylene group, a n-propylene group, an isopropylene group, a cyclopropylene group, a n-butylene group, an isobutylene group, a s-butylene group, a t-butylene group, a cyclobutylene group, a 1-methyl-cyclopropylene group, a 2-methyl-cyclopropylene group, a n-pentylene group, a 1-methyl-n-butylene group, a 2-methyl-n-butylene group, a 3-methyl-n-butylene group, a 1,1-dimethyl-n-propylene group, a 1,2-dimethyl-n-propylene group, a 2,2-dimethyl-n-propylene, a 1-ethyl-n-propylene group, a cyclopentylene group, a 1-methyl-cyclobutylene group, a 2-methyl-cyclobutylene group, a 3-methyl-cyclobutylene group, a 1,2-dimethyl-cyclopropylene group, a 2,3-dimethyl-cyclopropylene group, a 1-ethyl-cyclopropylene group, a 2-ethyl-cyclopropylene group, a n-hexylene group, a 1-methyl-n-pentylene group, a 2-methyl-n-pentylene group, a 3-methyl-n-pentylene group, a 4-methyl-n-pentylene group, a 1,1-dimethyl-n-butylene group, a 1,2-dimethyl-n-butylene group, a 1,3-dimethyl-n-butylene group, a 2,2-dimethyl-n-butylene group, a 2,3-dimethyl-n-butylene group, a 3,3-dimethyl-n-butylene group, a 1-ethyl-n-butylene group, a 2-ethyl-n-butylene group, a 1,1,2-trimethyl-n-propylene group, a 1,2,2-trimethyl-n-propylene group, a 1-ethyl-1-methyl-n-propylene group, a 1-ethyl-2-methyl-n-propylene group, a cyclohexylene group, a 1-methyl-cyclopentylene group, a 2-methyl-cyclopentylene group, a 3-methyl-cyclopentylene group, a 1-ethyl-cyclobutylene group, a 2-ethyl-cyclobutylene group, a 3-ethyl-cyclobutylene group, a 1,2-dimethyl-cyclobutylene group, a 1,3-dimethyl-cyclobutylene group, a 2,2-dimethyl-cyclobutylene group, a 2,3-dimethyl-cyclobutylene group, a 2,4-dimethyl-cyclobutylene group, a 3,3-dimethyl-cyclobutylene group, a 1-n-propyl-cyclopropylene group, a 2-n-propyl-cyclopropylene group, a 1-isopropyl-cyclopropylene group, a 2-isopropyl-cyclopropylene group, a 1,2,2-trimethyl-cyclopropylene group, a 1,2,3-trimethyl-cyclopropylene group, a 2,2,3-trimethyl-cyclopropylene group, a 1-ethyl-2-methyl-cyclopropylene group, a 2-ethyl-1-methyl-cyclopropylene group, a 2-ethyl-2-methyl-cyclopropylene group, a 2-ethyl-3-methyl-cyclopropylene group, a n-heptylene group, a n-octylene group, a n-nonylene group, and a n-decanylene group.

R¹ may be a C₁₋₁₀ alkoxy group.

Examples of the C₁₋₁₀ alkoxy group include a methoxy group, an ethoxy group, a n-propoxy group, an i-propoxy group, a n-butoxy group, an i-butoxy group, a s-butoxy group, a t-butoxy group, a n-pentoxy group, a 1-methyl-n-butoxy group, a 2-methyl-n-butoxy group, a 3-methyl-n-butoxy group, a 1,1-dimethyl-n-propoxy group, a 1,2-dimethyl-n-propoxy group, a 2,2-dimethyl-n-propoxy group, a 1-ethyl-n-propoxy group, a n-hexyloxy group, a 1-methyl-n-pentyloxy group, a 2-methyl-n-pentyloxy group, a 3-methyl-n-pentyloxy group, a 4-methyl-n-pentyloxy group, a 1,1-dimethyl-n-butoxy group, a 1,2-dimethyl-n-butoxy group, a 1,3-dimethyl-n-butoxy group, a 2,2-dimethyl-n-butoxy group, a 2,3-dimethyl-n-butoxy group, a 3,3-dimethyl-n-butoxy group, a 1-ethyl-n-butoxy group, a 2-ethyl-n-butoxy group, a 1,1,2-trimethyl-n-propoxy group, a 1,2,2-trimethyl-n-propoxy group, a 1-ethyl-1-methyl-n-propoxy group, a 1-ethyl-2-methyl-n-propoxy group, a n-heptyloxy group, a n-octyloxy group, and a n-nonyloxy group.

The unit structure represented by the formula (1-1) may be one kind or a combination of two or more kinds. For example, a copolymer having a plurality of unit structures in which Ar is the same kind may be used, and for example, a copolymer having a plurality of unit structures in which the type of Ar is different, such as a copolymer having a unit structure in which Ar contains a benzene ring and a unit structure in which Ar contains a naphthalene ring, is not excluded from the technical scope of the present application.

The phrase “optionally interrupted” means that in the case of a C₂₋₁₀ alkylene group, any carbon-carbon atom in the alkylene group is interrupted by a hetero atom (that is, an ether bond in the case of oxygen, and a sulfide bond in the case of sulfur), an ester bond, or an amide bond, and in the case of a carbon atom number of 1 (that is, a methylene group), either one of the carbons of the methylene group has a hetero atom (that is, an ether bond in the case of oxygen, and a sulfide bond in the case of sulfur), an ester bond, or an amide bond.

T¹ represents a C₁₋₁₀ alkylene group which may be interrupted by a single bond, an ether bond, an ester bond or an amide bond when n2=1, and is preferably a combination of an ether bond and a methylene group (that is, when “-T¹-(E)n2” in the formula (1-1) is a glycidyl ether group), a combination of an ester bond and a methylene group, or a combination of an amide bond and a methylene group.

The C₁₋₁₀ alkyl group which may be substituted with a hetero atom means that one or more hydrogen atoms of the C₁₋₁₀ alkyl group are substituted with a hetero atom (preferably a halogeno group).

L¹ represents a single bond or a C₁₋₁₀ alkylene group, and is represented by the following formula (1-2):

—CR²R³— Formula (1-2)
in the formula (1-2), R² and R³ each independently represent a hydrogen atom, a methyl group, an ethyl group, a n-propyl group, an i-propyl group, a cyclopropyl group, a n-butyl group, an i-butyl group, a s-butyl group, a t-butyl group, or a cyclobutyl group, and R² and R³ may be bonded to each other to form a C₃₋₆ ring. Among them, both R² and R³ are preferably hydrogen atoms (that is, —(CR²R³)— is a methylene group).

The halogeno group refers to halogen-X (F, Cl, Br, I) substituted with hydrogen.

E in the formula (1-1) is more preferably a group having an epoxy group.

The polymer in the fourth aspect is not particularly limited as long as it satisfies, for example, the unit structure of the formula (1-1). The polymer may be produced by a known method. Commercially available products may be used. Examples of the commercially available product include heat-resistant epoxy novolac resin EOCN (registered trademark) series (manufactured by Nippon Kayaku Co., Ltd., epoxy novolac resin D.E.N (registered trademark) series (manufactured by Dow Chemical Japan Ltd.).

The weight average molecular weight of the polymer in the fourth aspect is 100 or more, 500 to 200,000, 600 to 50,000, or 700 to 10,000.

Examples of the polymer in the fourth aspect include those having the following unit structure.

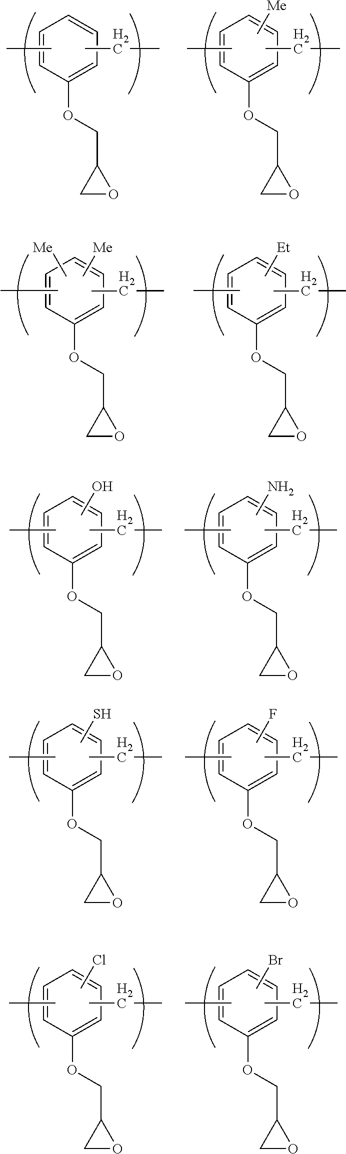

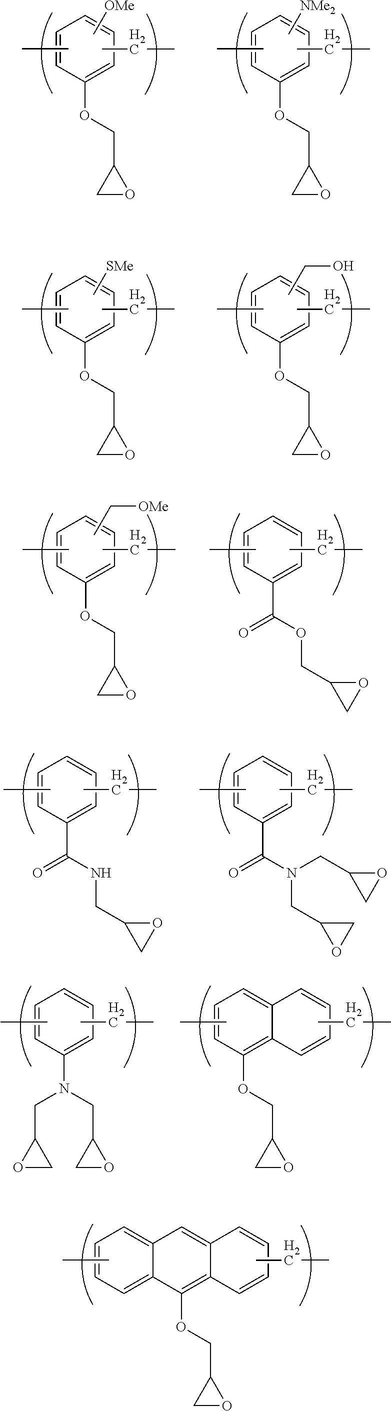

### (Resist Underlayer Film-Forming Composition)

The resist underlayer film-forming composition of the present invention contains:
(A) a compound represented by the above formula (A); and
(B) a solvent.

The protective film-forming composition of the present invention described above not only exhibits excellent resistance to a semiconductor wet etchant but also can be effectively used as a resist underlayer film-forming composition.

The description of terms related to the resist underlayer film-forming composition of the present invention is the same as the description content of the protective film-forming composition.

### (Protective Film, Resist Underlayer Film, Method for Producing Resist-Patterned Substrate, and Method for Manufacturing Semiconductor Device)

Hereinafter, a method for producing a resist-patterned substrate and a method for manufacturing a semiconductor device using the protective film-forming composition (resist underlayer film-forming composition) according to the present invention will be described.

A resist-patterned substrate according to the present invention can be produced by applying the above-described protective film-forming composition (resist underlayer film-forming composition) onto a semiconductor substrate and baking the composition.

Examples of the semiconductor substrate to which the protective film-forming composition (resist underlayer film-forming composition) of the present invention is applied include silicon wafers, germanium wafers, and compound semiconductor wafers such as gallium arsenide, indium phosphide, titanium oxide wafers, titanium nitride wafers, gallium nitride, indium nitride, aluminum nitride, and tungsten nitride.

When a semiconductor substrate having an inorganic film formed on a surface thereof is used, the inorganic film is formed by, for example, atomic layer deposition (ALD), chemical vapor deposition (CVD), reactive sputtering, ion plating, vacuum deposition, or spin coating (spin on glass: SOG). Examples of the inorganic film include a polysilicon film, a silicon oxide film, a silicon nitride film, a silicon oxynitride film, a boro-phospho silicate glass (BPSG) film, a titanium nitride film, a titanium oxynitride film, a tungsten nitride film, a gallium nitride film, and a gallium arsenide film. The semiconductor substrate may be a stepped substrate in which so-called vias (holes), trenches (grooves), and the like are formed. For example, the via has a substantially circular shape when viewed from the upper surface, the diameter of the substantially circular shape is, for example, 2 nm to 20 nm, and the depth is 50 nm to 500 nm, and the width of the trench (groove, recess of the substrate) is, for example, 2 nm to 20 nm, and the depth is 50 nm to 500 nm. Since the protective film-forming composition (resist underlayer film-forming composition) of the present invention has a small weight average molecular weight and average particle size of the compound contained in the composition, the composition can be embedded even in the stepped substrate as described above without defects such as voids. It is an important characteristic that there is no defect such as a void for the next process (wet etching/dry etching of semiconductor substrate, resist pattern formation) of semiconductor manufacturing.

The protective film-forming composition (resist underlayer film-forming composition) of the present invention is applied onto such a semiconductor substrate by an appropriate application method such as a spinner or a coater. Thereafter, the applied film is baked using a heating means such as a hot plate to form a protective film (resist underlayer film) as a baked product of the coating film. The baking conditions are appropriately selected from a baking temperature of 100° C. to 400° C. and a baking time of 0.3 minutes to 60 minutes. The baking temperature is preferably 120° C. to 350° C., and the baking time is 0.5 minutes to 30 minutes, more preferably 150° C. to 300° C., and the baking time is 0.8 minutes to 10 minutes. The thickness of the protective film formed is, for example, 0.001 μm to 10 μm, preferably 0.002 μm to 1 μm, and more preferably 0.005 μm to 0.5 μm. When the temperature during baking is lower than the above range, crosslinking becomes insufficient, and resistance of the protective film ((resist underlayer film) formed to a resist solvent or a basic hydrogen peroxide aqueous solution may be difficult to obtain. On the other hand, when the temperature during baking is higher than the above range, the protective film (resist underlayer film) may be decomposed by heat.

A resist film is formed on the protective film of the substrate with a protective film formed as described above, and then exposed and developed to form a resist pattern.

The exposure is performed through a mask (reticle) for forming a predetermined pattern, and for example, i-line, KrF excimer laser, ArF excimer laser, EUV (extreme ultraviolet ray), or EB (electron beam) is used. An alkaline developer is used for the development, and the conditions are appropriately selected from a development temperature of 5° C. to 50° C. and a development time of 10 seconds to 300 seconds. As the alkaline developer, for example, aqueous solutions of alkalis such as inorganic alkalis such as sodium hydroxide, potassium hydroxide, sodium carbonate, sodium silicate, sodium metasilicate, and ammonia water, first amines such as ethylamine and n-propylamine, second amines such as diethylamine and di-n-butylamine, third amines such as triethylamine and methyldiethylamine, alcoholamines such as dimethylethanolamine and triethanolamine, quaternary ammonium salts such as tetramethylammonium hydroxide, tetraethylammonium hydroxide, and choline, and cyclic amines such as pyrrole and piperidine can be used. Further, alcohols such as isopropyl alcohol and surfactants such as nonionic surfactants can be also added each in an appropriate amount to the aqueous solutions of alkalis, and used. Among them, preferred developers are quaternary ammonium salts, and more preferred are tetramethylammonium hydroxide and choline. Furthermore, a surfactant or the like can be added to these developers. A method of performing development with an organic solvent such as butyl acetate in place of the alkali developer to develop portions where the alkali dissolution rate of the photoresist is not improved can also be used.

Next, the protective film (resist underlayer film) is dry-etched through the formed resist pattern as a mask. At that time, when the inorganic film is formed on the surface of the semiconductor substrate used, the surface of the inorganic film is exposed, and when the inorganic film is not formed on the surface of the semiconductor substrate used, the surface of the semiconductor substrate is exposed.

Further, the semiconductor substrate is wet-etched and cleaned with a semiconductor wet etchant through the dry-etched protective film (resist underlayer film) (and also through the resist pattern when the resist pattern remains on the protective film/resist underlayer film) as a mask, thereby forming a desired pattern.

As the semiconductor wet etchant, a general chemical for etching a semiconductor wafer can be used, and for example, both an acidic substance and a basic substance can be used.

Examples of the acidic substance include hydrogen peroxide, hydrofluoric acid, ammonium fluoride, acidic ammonium fluoride, ammonium hydrogen fluoride, buffered hydrofluoric acid, hydrochloric acid, nitric acid, sulfuric acid, phosphoric acid, and mixed solutions thereof.

Examples of the basic substance include ammonia, sodium hydroxide, potassium hydroxide, sodium cyanide, potassium cyanide, and basic hydrogen peroxide solutions obtained by mixing an organic amine such as triethanolamine with a hydrogen peroxide solution to make the pH basic. Specific examples thereof include SC-1 (ammonia-hydrogen peroxide solution). In addition, those capable of making the pH basic, for example, those in which urea and a hydrogen peroxide solution are mixed to cause thermal decomposition of urea by heating to generate ammonia and finally make the pH basic, can also be used as a chemical for wet etching.

Among them, an acidic hydrogen peroxide solution or a basic hydrogen peroxide solution is preferable.

These chemicals may contain an additive such as a surfactant.

The use temperature of the semiconductor wet etchant is desirably 25° C. to 90° C., and more desirably 40° C. to 80° C. The wet etching time is desirably 0.5 minutes to 30 minutes, and more desirably 1 minute to 20 minutes.

### EXAMPLES

Hereinafter, the contents and effects of the present invention will be described in more detail by way of examples, but the present invention is not limited thereto.

The weight average molecular weight of the polymer synthesized in the following Examples of the present specification is a measurement result by gel permeation chromatography (hereinafter, abbreviated as GPC). For the measurement, a HLC-8320GPC apparatus manufactured by Tosoh Corporation is used, and measurement conditions and the like are as follows.
GPC column: Shodex [registered trademark] Asahipak [registered trademark] (Showa Denko K.K.)
Column temperature: 40° C.
Solvent: Tetrahydrofuran (THF)
Flow rate: 0.35 mL/min
Standard sample: Polystyrene (manufactured by Tosoh Corporation)

### Synthesis Example 1

A reaction flask was charged with 5.00 g of a triazinetrione-type epoxy resin (product name: TEPIC, manufactured by Nissan Chemical Corporation), 9.31 g of 3,4-dihydroxyhydrocinnamic acid, 0.43 g of tetrabutylphosphonium bromide and 58.95 g of propylene glycol monomethyl ether, and the mixture was heated and stirred at an internal temperature of 105° C. for 24 hours under a nitrogen atmosphere.

The obtained reaction product corresponded to the following formula (I-1), and the weight average molecular weight Mw measured in terms of polystyrene by GPC was 768.

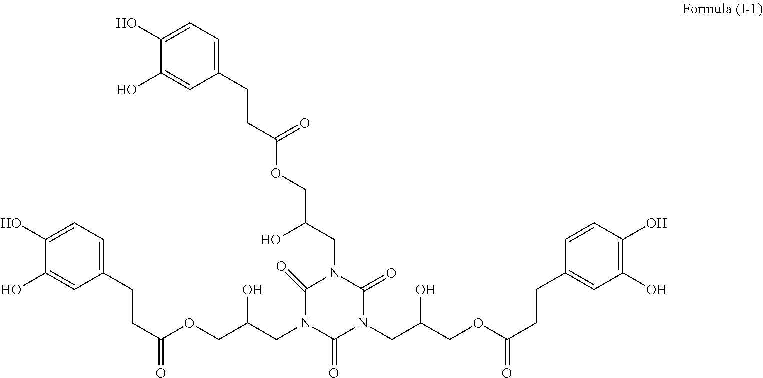 [SMILES: O=C(CCc1ccc(O)c(O)c1)OCC(O)Cn1c(=O)n(CC(O)COC(=O)CCc2ccc(O)c(O)c2)c(=O)n(CC(O)COC(=O)CCc2ccc(O)c(O)c2)c1=O]

### Synthesis Example 2

A reaction flask was charged with 3.00 g of a triazinetrione-type epoxy resin (product name: TEPIC, manufactured by Nissan Chemical Corporation), 2.61 g of cyanoacetic acid, 0.26 g of tetrabutylphosphonium bromide and 23.46 g of propylene glycol monomethyl ether, and the mixture was heated and stirred at an internal temperature of 80° C. for 24 hours under a nitrogen atmosphere. Subsequently, a solution prepared by dissolving 0.12 g of ammonium acetate and 4.24 g of 3,4-dihydroxybenzaldehyde in 17.42 g of propylene glycol monomethyl ether was added to the system, and the mixture was further heated and stirred at an internal temperature of 80° C. for 24 hours.

The obtained reaction product corresponded to the following formula (I-2), and the weight average molecular weight Mw measured in terms of polystyrene by GPC was 773.

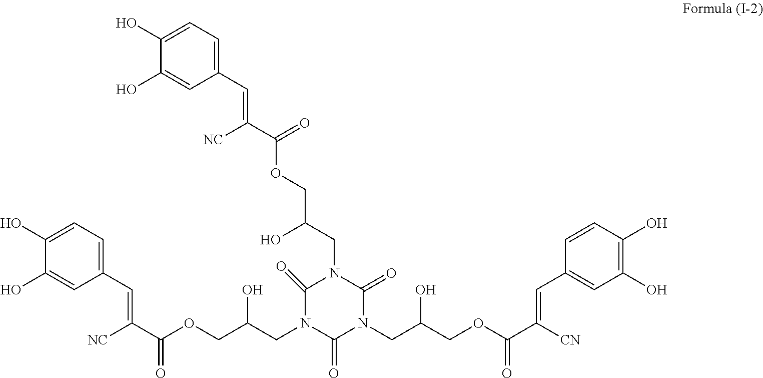 [SMILES: N#C/C(=C\c1ccc(O)c(O)c1)C(=O)OCC(O)Cn1c(=O)n(CC(O)COC(=O)/C(C#N)=C/c2ccc(O)c(O)c2)c(=O)n(CC(O)COC(=O)/C(C#N)=C/c2ccc(O)c(O)c2)c1=O]

### Synthesis Example 3

A reaction flask was charged with 3.00 g of a triazinetrione-type epoxy resin (product name: TEPIC, manufactured by Nissan Chemical Corporation), 2.61 g of cyanoacetic acid, 0.26 g of tetrabutylphosphonium bromide and 23.46 g of propylene glycol monomethyl ether, and the mixture was heated and stirred at an internal temperature of 80° C. for 24 hours under a nitrogen atmosphere. Subsequently, a solution prepared by dissolving 3.67 g of 4-hydroxybenzaldehyde in 14.68 g of propylene glycol monomethyl ether was added to the system, and the mixture was further heated and stirred at an internal temperature of 80° C. for 24 hours.

The obtained reaction product corresponded to the following formula (I-3), and the weight average molecular weight Mw measured in terms of polystyrene by GPC was 655.

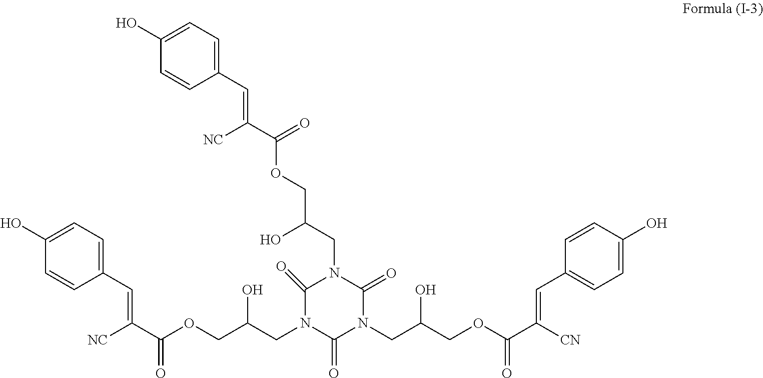 [SMILES: N#C/C(=C\c1ccc(O)cc1)C(=O)OCC(O)Cn1c(=O)n(CC(O)COC(=O)/C(C#N)=C/c2ccc(O)cc2)c(=O)n(CC(O)COC(=O)/C(C#N)=C/c2ccc(O)cc2)c1=O]

### Example 1

To 2.875 g of a solution of the reaction product corresponding to the above formula (I-1) (solid content: 16.7 mass %), 0.096 g of tetramethoxymethyl glycoluril (trade name: POWDER LINK [registered trademark] 1174, manufactured by Nihon Cytec Industries Inc.) as a crosslinking agent, 0.024 g of pyridinium-trifluoromethanesulfonate as a crosslinking catalyst, 0.001 g of MEGAFACE R-30N (manufactured by DIC Corporation, trade name) as a surfactant, and 7.00 g of propylene glycol monomethyl ether were added to prepare a solution of a protective film-forming composition.

### Example 2

To 2.875 g of a solution of the reaction product corresponding to the above formula (I-1) (solid content: 16.7 mass %), 0.096 g of tetramethoxymethyl glycoluril (trade name: POWDER LINK [registered trademark] 1174, manufactured by Nihon Cytec Industries Inc.) as a crosslinking agent, 0.024 g of pyridinium-p-toluenesulfonate as a crosslinking catalyst, 0.001 g of MEGAFACE R-30N (manufactured by DIC Corporation, trade name) as a surfactant, and 7.00 g of propylene glycol monomethyl ether were added to prepare a solution of a protective film-forming composition.

### Example 3

To 2.875 g of a solution of the reaction product corresponding to the above formula (I-1) (solid content: 16.7 mass %), 0.096 g of tetramethoxymethyl glycoluril (trade name: POWDER LINK [registered trademark] 1174, manufactured by Nihon Cytec Industries Inc.) as a crosslinking agent, 0.024 g of pyridinium-p-phenolsulfonate as a crosslinking catalyst, 0.001 g of MEGAFACE R-30N (manufactured by DIC Corporation, trade name) as a surfactant, and 7.00 g of propylene glycol monomethyl ether were added to prepare a solution of a protective film-forming composition.

### Example 4

To 2.875 g of a solution of the reaction product corresponding to the above formula (I-1) (solid content: 16.7 mass %), 0.096 g of 3,3′,5,5′-tetrakis(methoxymethyl)-4,4′-dihydroxybiphenyl (product name: TMOM-BP, manufactured by Honshu Chemical Industry Co., Ltd.) as a crosslinking agent, 0.024 g of pyridinium-trifluoromethanesulfonate as a crosslinking catalyst, 0.001 g of MEGAFACE R-30N (manufactured by DIC Corporation, trade name) as a surfactant, and 7.00 g of propylene glycol monomethyl ether were added to prepare a solution of a protective film-forming composition.

### Example 5

To 2.875 g of a solution of the reaction product corresponding to the above formula (I-1) (solid content: 16.7 mass %), 0.096 g of 3,3′,5,5′-tetrakis(methoxymethyl)-4,4′-dihydroxybiphenyl (product name: TMOM-BP, manufactured by Honshu Chemical Industry Co., Ltd.) as a crosslinking agent, 0.024 g of pyridinium-p-toluenesulfonate as a crosslinking catalyst, 0.001 g of MEGAFACE R-30N (manufactured by DIC Corporation, trade name) as a surfactant, and 7.00 g of propylene glycol monomethyl ether were added to prepare a solution of a protective film-forming composition.

### Example 6

To 2.875 g of a solution of the reaction product corresponding to the above formula (I-1) (solid content: 16.7 mass %), 0.096 g of 3,3′,5,5′-tetrakis(methoxymethyl)-4,4′-dihydroxybiphenyl (product name: TMOM-BP, manufactured by Honshu Chemical Industry Co., Ltd.) as a crosslinking agent, 0.024 g of pyridinium-p-phenolsulfonate as a crosslinking catalyst, 0.001 g of MEGAFACE R-30N (manufactured by DIC Corporation, trade name) as a surfactant, and 7.00 g of propylene glycol monomethyl ether were added to prepare a solution of a protective film-forming composition.

### Example 7

To 4.149 g of a solution of the reaction product corresponding to the above formula (I-2) (solid content: 17.3 mass %), 0.144 g of 3,3′,5,5′-tetrakis(methoxymethyl)-4,4′-dihydroxybiphenyl (product name: TMOM-BP, manufactured by Honshu Chemical Industry Co., Ltd.) as a crosslinking agent, 0.036 g of pyridinium-trifluoromethanesulfonate as a crosslinking catalyst, 0.001 g of MEGAFACE R-30N (manufactured by DIC Corporation, trade name) as a surfactant, and 10.67 g of propylene glycol monomethyl ether were added to prepare a solution of a protective film-forming composition.

### Example 8

To 4.490 g of a solution of an acrylic resin of a chemical-resistant protective film-forming composition represented by the following formula (I-4) (solid content: 20.0 mass %), 1.073 g of a solution of the reaction product corresponding to the above formula (I-1) (solid content: 16.7 mass %) as an additive, 0.179 g of tetramethoxymethyl glycoluril (trade name: POWDER LINK [registered trademark]1174, manufactured by Nihon Cytec Industries Inc.) as a crosslinking agent, 0.045 g of pyridinium-trifluoromethanesulfonate as a crosslinking catalyst, 0.001 g of MEGAFACE R-30N (manufactured by DIC Corporation, trade name) as a surfactant, 9.50 g of ethyl lactate, and 4.71 g of propylene glycol monomethyl ether were added to prepare a solution of a protective film-forming composition.

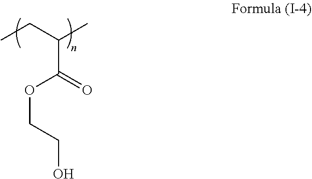

### Example 9

To 4.491 g of a solution of an acrylic resin of the chemical-resistant protective film-forming composition represented by the above formula (I-4) (solid content: 20.0 mass %), 1.033 g of a solution of the reaction product corresponding to the above formula (I-2) (solid content: 17.3 mass %) as an additive, 0.179 g of tetramethoxymethyl glycoluril (trade name: POWDER LINK [registered trademark]1174, manufactured by Nihon Cytec Industries Inc.) as a crosslinking agent, 0.045 g of pyridinium-trifluoromethanesulfonate as a crosslinking catalyst, 0.001 g of MEGAFACE R-30N (manufactured by DIC Corporation, trade name) as a surfactant, 9.50 g of ethyl lactate, and 4.76 g of propylene glycol monomethyl ether were added to prepare a solution of a protective film-forming composition.

### Example 10

To 3.909 g of a solution of an acrylic resin of the chemical-resistant protective film-forming composition represented by the following formula (I-5) (solid content: 30.2 mass %), 0.708 g of a solution of the reaction product corresponding to the above formula (I-1) (solid content: 16.7 mass %) as an additive, 0.001 g of MEGAFACE R-30N (manufactured by DIC Corporation, trade name) as a surfactant, 5.02 g of propylene glycol monomethyl ether, and 10.36 g of propylene glycol monomethyl ether acetate were added to prepare a solution of a protective film-forming composition.

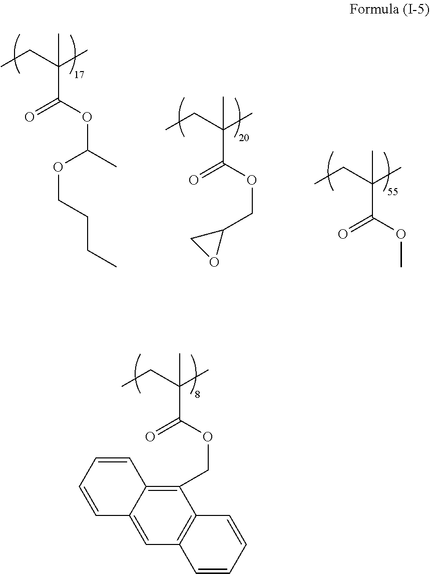

### Example 11

To 3.909 g of a solution of an acrylic resin of the chemical-resistant protective film-forming composition represented by the above formula (I-5) (solid content: 30.2 mass %), 0.681 g of a solution of the reaction product corresponding to the above formula (I-2) (solid content: 17.3 mass %) as an additive, 0.001 g of MEGAFACE R-30N (manufactured by DIC Corporation, trade name) as a surfactant, 5.05 g of propylene glycol monomethyl ether, and 10.36 g of propylene glycol monomethyl ether acetate were added to prepare a solution of a protective film-forming composition.

### Comparative Example 1

To 5.187 g of a solution of the reaction product corresponding to the above formula (I-3) (solid content: 13.9 mass %), 0.144 g of 3,3′,5,5′-tetrakis(methoxymethyl)-4,4′-dihydroxybiphenyl (product name: TMOM-BP, manufactured by Honshu Chemical Industry Co., Ltd.) as a crosslinking agent, 0.036 g of pyridinium-trifluoromethanesulfonate as a crosslinking catalyst, 0.001 g of MEGAFACE R-30N (manufactured by DIC Corporation, trade name) as a surfactant, and 10.67 g of propylene glycol monomethyl ether were added to prepare a solution of a protective film-forming composition.

### Comparative Example 2

To 5.209 g of a solution of an acrylic resin of the protective film-forming composition represented by the above formula (I-4) (solid content: 20.0 mass %), 0.208 g of tetramethoxymethyl glycoluril (trade name: POWDER LINK [registered trademark] 1174, manufactured by Nihon Cytec Industries Inc.) as a crosslinking agent, 0.052 g of pyridinium-trifluoromethanesulfonate as a crosslinking catalyst, 0.001 g of MEGAFACE R-30N (manufactured by DIC Corporation, trade name) as a surfactant, 8.92 g of ethyl lactate, and 5.61 g of propylene glycol monomethyl ether were added to prepare a solution of a protective film-forming composition.

### Comparative Example 3

To 4.299 g of a solution of an acrylic resin of the protective film-forming composition represented by the above formula (I-5) (solid content: 30.2 mass %), 0.001 g of MEGAFACE R-30N (manufactured by DIC Corporation, trade name) as a surfactant, 5.61 g of propylene glycol monomethyl ether, and 10.09 g of propylene glycol monomethyl ether acetate were added to prepare a solution of a protective film-forming composition.

### (Test of Resistance to Resist Solvents)

The protective film-forming compositions prepared in Examples 1 to 11 and Comparative Examples 1 to 3 were each applied (spin-coated) onto a silicon wafer with a spin coater.

The coated silicon wafer was heated on a hot plate at 220° C. for 1 minute to form a coating (protective film) with a film thickness of 150 nm. Next, in order to confirm resistance of the protective film to a resist solvent, the silicon wafer on which the protective film had been formed was immersed for 1 minute in a solvent containing propylene glycol monomethyl ether and propylene glycol monomethyl ether acetate mixed at a mass ratio of 7:3, spin-dried, and then baked at 100° C. for 30 seconds. The film thickness of the protective film before and after the immersion in the mixed solvent was measured with a light interference film thickness meter (product name: NanoSpec 6100, manufactured by Nanometrics Japan Co., Ltd.).

The resist solvent resistance was evaluated by calculating the film thickness reduction rate (%) of the protective film removed by solvent immersion from the equation: ((film thickness before solvent immersion)−(film thickness after solvent immersion))/(film thickness before solvent immersion)×100. The results are shown in Table 1 below. When the film thickness reduction rate is about 1% or less, it can be said that the film has sufficient resist solvent resistance.

TABLE 1

| | Film thickness |
|---|---|
| | reduction rate |
| Example 1 | 0.20% |
| Example 2 | 0.10% |
| Example 3 | 0.20% |
| Example 4 | −0.20% |
| Example 5 | 0.10% |
| Example 6 | 0.10% |
| Example 7 | 0.40% |
| Example 8 | 0.10% |
| Example 9 | −0.10% |
| Example 10 | 0.10% |
| Example 11 | −0.20% |
| Comparative | 0.70% |
| Example 1 | |
| Comparative | 0.00% |
| Example 2 | |
| Comparative | 0.10% |
| Example 3 | |

From the above results, the protective film-forming compositions of Examples 1 to 11 and Comparative Examples 1 to 3 had very small changes in film thickness even after immersion in the resist solvent. Therefore, the protective film-forming compositions of Examples 1 to 11 have sufficient resist solvent resistance to function as protective films.

### (Test of Resistance to Basic Hydrogen Peroxide Aqueous Solution)

As an evaluation of resistance to a basic hydrogen peroxide solution, each of the protective film-forming compositions prepared in Examples 1 to 6, Examples 8 to 11, and Comparative Examples 2 to 3 was applied to a 50 nm thick titanium nitride (TiN)-deposited substrate, and heated at 220° C. for 1 minute to form a protective film with a film thickness of 150 nm. Next, 28% aqueous ammonia, 33% hydrogen peroxide and water were mixed at a mass ratio of 1:4:20 to prepare a basic hydrogen peroxide solution. The TiN-deposited substrate coated with the protective film-forming composition was immersed in this basic hydrogen peroxide solution heated to 50° C., and the time was measured from immediately after the immersion until the protective film was peeled off from the substrate (peeling time). The results of the test of resistance to a basic hydrogen peroxide solution are shown in Table 2 below. It can be said that the longer the peeling time, the higher the resistance to the wet etchant using the basic hydrogen peroxide solution.

### (Test of Resistance to Hydrogen Peroxide Solution)

As an evaluation of resistance to an acidic hydrogen peroxide solution, each of the protective film-forming compositions prepared in Examples 1 to 11 and Comparative Example 1 was applied to a 50 nm thick TiN-deposited substrate, and heated at 220° C. for 1 minute to form a protective film with a film thickness of 150 nm. Next, the TiN-deposited substrate coated with the protective film-forming composition was immersed in the 20 mass % hydrogen peroxide solution heated to 70° C., and the time was measured from immediately after the immersion until the protective film was damaged. The results of the test of resistance to a hydrogen peroxide solution are shown in Table 2. It can be said that the longer the time until the damage occurs, the higher the resistance to the wet etchant using the hydrogen peroxide solution.

TABLE 2

| | Peeling time of protective | Time until protective film for |
|---|---|---|
| | film against basic | 20 mass % hydrogen peroxide |
| | hydrogen peroxide solution | solution was damaged |
| Example 1 | 830 sec | 200 sec or more |
| Example 2 | 385 sec | 200 sec or more |
| Example 3 | 708 sec | 200 sec or more |
| Example 4 | 1297 sec | 200 sec or more |
| Example 5 | 496 sec | 200 sec or more |
| Example 6 | 956 sec | 200 sec or more |
| Example 7 | — | 200 sec or more |
| Example 8 | 330 sec | 200 sec or more |
| Example 9 | 232 sec | 200 sec or more |
| Example 10 | 807 sec | 200 sec or more |
| Example 11 | 287 sec | 200 sec or more |
| Comparative | — | 100 sec or less |
| Example 1 | | |
| Comparative | 118 sec | — |
| Example 2 | | |
| Comparative | 229 sec | — |
| Example 3 | | |

From the above results, when comparing Examples 1 to 6 and Examples 8 to 11 using a reaction product having a structure containing at least one set of catechol groups in the molecule at the terminal with Comparative Examples 2 to 3 not using such a reaction product, the peeling time of the protective film against the basic hydrogen peroxide solution was longer in Examples 1 to 6 and Examples 8 to 11. Similarly, when comparing Examples 1 to 11 with Comparative Example 1, the time until the protective film for the hydrogen peroxide solution was damaged was longer in Examples 1 to 11. That is, from the results of Examples 1 to 11, by selecting and adopting the reaction product having a structure containing at least one set of catechol groups in the molecule at the terminal, it was possible to obtain good resistance to a wet etchant using a basic hydrogen peroxide solution, a hydrogen peroxide solution, or both, as compared with Comparative Examples 1 to 3 in which such a reaction product is not selected and adopted. Further, from the results of Examples 1 to 6, it can be said that by selecting a phenoplast crosslinking agent as a crosslinking agent and a catalyst that generates a superacid as a crosslinking catalyst, good resistance to a wet etchant using a basic hydrogen peroxide solution is exhibited. Therefore, Examples 1 to 11 exhibit better chemical resistance to a basic hydrogen peroxide solution, a hydrogen peroxide solution, or both, as compared with Comparative Examples 1 to 3, and thus are useful as protective films against a semiconductor wet etchant.

### INDUSTRIAL APPLICABILITY

The protective film-forming composition according to the present invention is excellent in resistance when a wet etchant is applied to substrate processing while exhibiting good resistance to a resist solvent which is mainly an organic solvent, and therefore provides a protective film which is less damaged on the protective film during substrate processing. The resist underlayer film-forming composition according to the present invention is excellent in resistance when a wet etchant is applied to substrate processing.

1. A protective film-forming composition against a semiconductor wet etchant, comprising:
(A) a compound represented by the following formula (A); and
(B) a solvent, 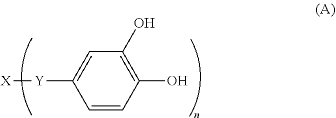
in the formula (A), n represents an integer of 1 to 10, and when n is 2, X represents a sulfinyl group, a sulfonyl group, an ether group or a C₂₋₅₀ divalent organic group, and when n is an integer other than 2, X represents a C₂₋₅₀ n-valent organic group, Y represents —CH₂CH(OH)CH₂OC(═O)CH₂(CH₂)ₜ—, —CH₂CH(OH)CH₂OC(═O)C(CN)(═CH)—, and t represents an integer of 1 to 6.

2. The protective film-forming composition according to claim 1, wherein in the formula (A), when the X is a C₂₋₅₀ divalent organic group, the X is a divalent organic group represented by the following formula (A-1), and when the X is a C₂₋₅₀ n-valent organic group other than a C₂₋₅₀ divalent organic group, the X is an n-valent organic group represented by the following formula (A-2), 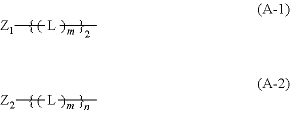
in the formula (A-1), Z₁ represents a C₁₋₆ alkylene group or a divalent organic group containing a ring selected from the group consisting of an aromatic ring optionally having a substituent, an aliphatic ring optionally having a substituent, and a heterocyclic ring optionally having a substituent, or a divalent organic group containing the ring and a C₁₋₆ alkylene group, m represents 0 or 1, and L represents —O— or —C(═O)—O—, and
in the formula (A-2), Z₂ represents an n-valent organic group containing a ring selected from the group consisting of an aromatic ring optionally having a substituent, an aliphatic ring optionally having a substituent, and a heterocyclic ring optionally having a substituent, or an n-valent organic group containing the ring and a C₁₋₆ alkylene group, m represents 0 or 1, and L represents —O— or —C(═O)—O—.

3. The protective film-forming composition according to claim 1, wherein the protective film-forming composition further comprises at least one of (C) a crosslinking agent, (D) a crosslinking catalyst, and (E) a surfactant.

4. The protective film-forming composition according to claim 1, wherein the protective film-forming composition further comprises (F) a compound or polymer containing a (meth)acryloyl group, a styrene group, a phenolic hydroxy group, an ether group, an epoxy group, or an oxetanyl group.

5. The protective film-forming composition according to claim 4, wherein the protective film-forming composition further comprises (G) a polymer having a repeating structural unit represented by the following formula (G): 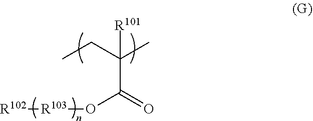
in the formula (G), R¹⁰¹ represents a hydrogen atom or a methyl group, R¹⁰² represents a group selected from the following formulas (g-1) to (g-3), a C₁₋₄ alkyl group optionally interrupted by oxygen, an aryl group optionally substituted, or a hydroxy group, R¹⁰³ represents a C₁₋₄ alkylene group, and n represents 0 or 1, and in the formulas (g-1) to (g-3), * represents a bond,

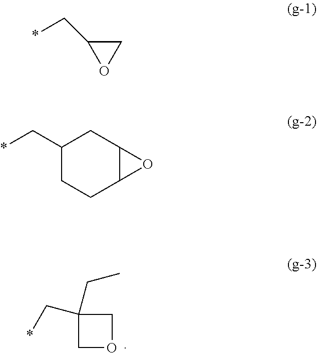

6. The protective film-forming composition according to claim 4, further comprising (J) a compound (J) or a polymer (J) containing a cyclic ether having a 3-membered ring structure or a 4-membered ring structure.

7. A protective film against a semiconductor wet etchant, which is a baked product of a coating film comprising the protective film-forming composition according to claim 1.

8. A resist underlayer film-forming composition, comprising:
(A) a compound represented by the following formula (A); and
(B) a solvent, 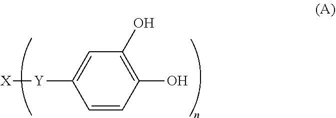
in the formula (A), n represents an integer of 1 to 10, and when n is 2, X represents a sulfinyl group, a sulfonyl group, an ether group or a C₂₋₅₀ divalent organic group, and when n is an integer other than 2, X represents a C₂₋₅₀ n-valent organic group, Y represents —CH₂CH(OH)CH₂OC(═O)CH₂(CH₂)ₜ—, —CH₂CH(OH)CH₂OC(═O)C(CN)(═CH)—, and t represents an integer of 1 to 6.

9. The resist underlayer film-forming composition according to claim 8, wherein in the formula (A), when the X is a C₂₋₅₀ divalent organic group, the X is a divalent organic group represented by the following formula (A-1), and when the X is a C₂₋₅₀ n-valent organic group other than a C₂₋₅₀ divalent organic group, the X is an n-valent organic group represented by the following formula (A-2), 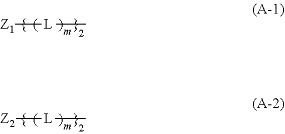
in the formula (A-1), Z₁ represents a C₁₋₆ alkylene group or a divalent organic group containing a ring selected from the group consisting of an aromatic ring optionally having a substituent, an aliphatic ring optionally having a substituent, and a heterocyclic ring optionally having a substituent, or a divalent organic group containing the ring and a C₁₋₆ alkylene group, m represents 0 or 1, and L represents —O— or —C(═O)—O—, and
in the formula (A-2), Z₂ represents an n-valent organic group containing a ring selected from the group consisting of an aromatic ring optionally having a substituent, an aliphatic ring optionally having a substituent, and a heterocyclic ring optionally having a substituent, or an n-valent organic group containing the ring and a C₁₋₆ alkylene group, m represents 0 or 1, and L represents —O— or —C(═O)—O—.

10. A resist underlayer film, which is a baked product of a coating film comprising the resist underlayer film-forming composition according to claim 8.

11. A method for producing a substrate with a protective film, comprising forming a protective film by applying the protective film-forming composition according to claim 1 onto a stepped semiconductor substrate and baking the composition, wherein the substrate is used for producing a semiconductor.

12. A method for producing a resist-patterned substrate, comprising applying the protective film-forming composition according to claim 1 onto a semiconductor substrate and baking the composition to form a protective film as a resist underlayer film; and forming a resist film on the protective film and then exposing and developing the composition to form a resist pattern, wherein the substrate is used for producing a semiconductor.

13. A method for manufacturing a semiconductor device, comprising forming a protective film with the protective film-forming composition according to claim 1 on a semiconductor substrate optionally having an inorganic film formed on a surface thereof, forming a resist pattern on the protective film, dry-etching the protective film through the resist pattern as a mask to expose a surface of the inorganic film or of the semiconductor substrate, and wet etching and cleaning the inorganic film or the semiconductor substrate with a semiconductor wet etchant through the dry-etched protective film as a mask.

14. A method for manufacturing a semiconductor device, comprising forming a resist underlayer film with the resist underlayer film-forming composition according to claim 8 on a semiconductor substrate optionally having an inorganic film formed on a surface thereof, forming a resist pattern on the resist underlayer film, dry-etching the resist underlayer film through the resist pattern as a mask to expose a surface of the inorganic film or of the semiconductor substrate, and etching the inorganic film or the semiconductor substrate through the dry-etched resist underlayer film as a mask.

15. A method for producing a resist-patterned substrate, comprising applying the resist underlayer film-forming composition according to claim 8 onto a semiconductor substrate and baking the composition to form a protective film as a resist underlayer film; and forming a resist film on the protective film and then exposing and developing the composition to form a resist pattern, wherein the substrate is used for producing a semiconductor.
